(19) United States
(12) Patent Application Publication
(10) Pub. No.: US 2026/0296903 A1
(43) Pub. Date: Oct. 1, 2026

(54) ALUMINUM-CONTAINING MOLECULAR SIEVES OF TON FRAMEWORK TYPE AND METHODS OF MAKING AND USE

(71) Applicant: EXXONMOBIL TECHNOLOGY AND ENGINEERING COMPANY, Spring, TX (US)

(72) Inventors: Trong D. PHAM, Flemington, NJ (US); Emiel De Smit, Brussels (BE); Michael P. Lanci, Flemington, NJ (US); Mary S. Abdulkarim, Palisades Park, NJ (US)

(21) Appl. No.: 19/634,398

(22) Filed: Mar. 31, 2026

Related U.S. Application Data
(60) Provisional application No. 63/781,959, filed on Apr. 1, 2025.

(51) Int. Cl. C01B 39/02; C10G 50/00

(52) U.S. Cl. CPC C01B 39/026; C10G 50/00; C01P 2002/72; C01P 2004/03; C01P 2004/64

(57) ABSTRACT

The present disclosure relates to a method of making aluminum-containing molecular sieves of TON framework type. The present disclosure further relates to small crystal forms of aluminum-containing molecular sieves of TON framework type, and uses thereof. It has been found that the use of imidazolium cations selected from 1,3-dimethylimidazolium cations and 1-ethyl-3-methylimidazolium (or 1-methyl-3-ethylimidazolium) cations as structure directing agents (SDAs) allows the preparation of aluminum-containing molecular sieves of TON framework type, in particular in the form of small crystals. Methods of making aluminum-containing molecular sieves of TON framework type using said SDAs are provided, along with corresponding compositions.

### CROSS REFERENCE TO RELATED APPLICATION

This Non-Provisional patent application claims priority to U.S. Provisional Patent Application No. 63/781,959, filed Apr. 1, 2025, and titled “Aluminum-Containing Molecular Sieves Of Ton Framework Type And Methods Of Making And Use,” the entire contents of which is incorporated herein by reference.

### TECHNICAL FIELD

The present disclosure relates to a method of making aluminum-containing molecular sieves of TON framework type. The present disclosure also relates to small crystal forms of aluminum-containing molecular sieves of TON framework type and uses thereof.

### BACKGROUND OF THE INVENTION

Molecular sieve materials, both natural and synthetic, may be used as adsorbents and have catalytic properties for hydrocarbon conversion reactions. Certain molecular sieves, such as zeolites, AlPOs, and mesoporous materials, are ordered, porous crystalline materials having a definite crystalline structure as determined by X-ray diffraction (XRD). Certain molecular sieves are ordered and produce specific identifiable XRD patterns. Within certain molecular sieve materials there may be a large number of cavities, which may be interconnected by a number of channels or pores. These cavities and pores are uniform in size within a specific molecular sieve material. Because the dimensions of these pores are such as to accept for adsorption molecules of certain dimensions while rejecting those of larger dimensions, these materials have come to be known as “molecular sieves” and are utilized in a variety of industrial processes, e.g., cracking, hydrocracking, disproportionation, alkylation, oligomerization, and isomerization.

Molecular sieves that find application in catalysis and adsorption include any of the naturally occurring or synthetic crystalline molecular sieves. These zeolites and their isotypes are classified by the Structure Commission of the International Zeolite Association according to the rules of the IUPAC Commission on Zeolite Nomenclature. According to this classification, framework type zeolites and other crystalline microporous molecular sieves, for which a structure has been established, are assigned a three letter code and are described in the “Atlas of Zeolite Framework Types”, eds. Ch. Baerlocher, L. B. McCusker, and D. H. Olson, Elsevier, Sixth Edition, 2007. These zeolites and their isotypes are also described in http://America.iza-structure.org/IZA-SC/ftc_table.php.

The idealized inorganic framework structure of zeolites is a framework of silicate in which all tetrahedral atoms are connected by oxygen atoms with the four next-nearest tetrahedral atoms. The term “silicate”, as used herein, refers to a substance containing at least silicon and oxygen atoms that are alternately bonded to each other (i.e., —O—Si—O—Si—), and optionally including other atoms within the inorganic framework structure, including atoms such as boron, aluminum, or other metals (e.g., transition metals, such as titanium, vanadium, or zinc). Atoms other than silicon and oxygen in the framework silicate occupy a portion of the lattice sites otherwise occupied by silicon atoms in an ‘all-silica’ framework silicate. Thus, the term “framework silicate” as used herein refers to an atomic lattice comprising any of a silicate, borosilicate, gallosilicate, ferrisilicate, aluminosilicate, titanosilicate, zincosilicate, vanadosilicate, or the like.

The structure of the framework silicate within a given zeolite determines the size of the pores or channels that are present therein. The pore or channel size may determine the types of processes for which a given zeolite is applicable. Currently, greater than 200 unique zeolite framework silicate structures are known and recognized by the Structure Commission of the International Zeolite Association, thereby defining a range of pore geometries and orientations.

The framework silicates of zeolites or molecular sieves are commonly characterized in terms of their ring size, wherein the ring size refers to the number of silicon atoms (or alternative atoms, such as those listed above) that are tetrahedrally coordinated with oxygen atoms in a loop to define a pore or channel within the interior of the zeolite. For example, an “8-ring” zeolite refers to a zeolite having pores or channels defined by 8 alternating tetrahedral atoms and 8 oxygen atoms in a loop. The pores or channels defined within a given zeolite may be symmetrical or asymmetrical depending upon various structural constrains that are present in the particular framework silicate.

Zeolites can be classified as having small, medium, large, and extra-large pore structures for pore windows delimited by 8, 10, 12, and more than 12 T-atoms, respectively. Extra-large pore zeolites (>12R) include, for example, AET (14R, e.g., ALPO-8), SFN (14R, e.g., SSZ-59), VFI (18R, e.g., VPI-5), CLO (20R, e.g., cloverite), and ITV (30R, e.g., ITQ-37) framework type zeolites. Extra-large pore zeolites generally have a free pore diameter of larger than about 0.8 nm. Large pore zeolites (12R) include, for example, LTL, MAZ, FAU, EMT, OFF, MTW, *BEA, MOR, and SFS framework type zeolites, e.g., mazzite, offretite, zeolite L, zeolite Y, zeolite X, omega, ZSM-2, ZSM-12, zeolite T, Beta, and SSZ-56. Large pore zeolites generally have a free pore diameter of 0.6 to 0.8 nm. Medium (or intermediate) pore size zeolites (10R) include, for example, MFI, MEL, *MRE, EUO, MTT, MFS, AEL, AFO, HEU, FER, MWW, and TON framework type zeolites, e.g., ZSM-5, ZSM-11, ZSM-48, ZSM-22, ZSM-23, ZSM-35, MCM-22, silicalite-1, and silicalite-2. Medium pore size zeolites generally have a free pore diameter of 0.45 to 0.6 nm. Small pore size zeolites (8R) include, for example, CHA, RTH, ERI, KFI, LEV, and LTA framework type zeolites, e.g., ZK-4, SAPO-34, SAPO-35, ZK-14, SAPO-42, ZK-21, ZK-22, ZK-5, ZK-20, zeolite A, chabazite, and ALPO-17. Small pore size zeolites generally have a free pore diameter of 0.3 to 0.45 nm.

Synthesis of molecular sieve materials typically involves hydrothermal crystallization from a synthesis mixture comprising sources of all the elements present in the molecular sieve (or zeolite) such as sources of silica but also of alumina etc. In many cases a structure directing agent (SDA) is also present. Structure directing agents are compounds which are believed to promote the formation of molecular sieves and which are thought to act as templates around which certain molecular sieve structures can form and which thereby promote the formation of the desired molecular sieve. Various compounds have been used as structure directing agents including various types of quaternary ammonium cations. Typically, molecular sieve (or zeolite) crystals form around structure directing agents with the structure directing agent occupying pores in the molecular sieve once crystallization is complete. The “as-synthesized” (or “as-made”) molecular sieve will therefore contain the structure directing agent in its pores so that, following crystallization, the “as-synthesized” molecular sieve is usually subjected to a treatment step such as a calcination step to remove the structure directing agent.

For instance, molecular sieves of TON framework type are crystalline materials having one-dimensional 10-membered ring channels. Examples of molecular sieves of TON framework type include ISI-1, KZ-2, Nu-10, Theta-1 and ZSM-22. These materials are of significant commercial importance because of their activity as catalysts, for instance in dewaxing of paraffinic hydrocarbons.

EP 0 057 049 B2 discloses the hydrothermal synthesis of Theta-1 aluminosilicate zeolite having a SiO₂:Al₂O₂ molar ratio of at least 10, using an organic nitrogen containing base, such as diethanolamine, as structure directing agent. The crystals exhibited rod-like morphology, being about 0.1 μm in diameter and about 2 μm in length (S. A. I. Barri et al., Nature, Vol. 312, 533-534 (1984)).

EP 0 065 400 B1 discloses the hydrothermal synthesis of Nu-10 zeolite having a XO₂:Y₂O₃ molar ratio in the range 20 to 5000, X being silicon or germanium and Y being aluminium, iron, chromium, vanadium, molybdenum, arsenic, antimony, manganese, gallium or boron, using a polyalkylene polyamine as structure directing agent. The crystals were shown to be needles having an aspect ratio of at least 10 and with lengths varying from 0.5 to 1 μm, from 1 to 1.5 μm, or from 2 to 3 μm.

L. M. Parker et al., Zeolites, 1983, Vol 3, 8-10, discloses the hydrothermal synthesis and the properties of zeolite KZ-2, using diethylamine, 1-aminobutane, 1.4-diaminohexane, or 2,2′-diaminodiethylamine as structure directing agent. The pure silica KZ-2 crystals were rectangular rod-shaped with a size of about 0.5×0.5×2-3 μm, the crystal size decreasing when the aluminium content increased, the crystal size being halved and the crystals less well defined at the maximum Al₂O₃ concentration.

EP 0 102 716 B1 discloses the hydrothermal synthesis of ZSM-22 zeolite, which appears to have the same crystal structure as Nu-10, and its use in xylene isomerization. ZSM-22 zeolite with a SiO₂:Al₂O₃ molar ratio of 20 to ∞ was synthesized using an alkane diamine as structure directing agent. According to G. T. Kokotailo et al., Zeolites, 1985, Vol 5, 349-351, which discusses the framework topology of ZSM-22, single crystals of ZSM-22 are typically acicular with dimensions 0.2×0.2×1 μm.

Similarly, U.S. Pat. No. 4,902,406 discloses the synthesis of ZSM-22 zeolite from a synthesis mixture having a SiO₂:Al₂O₃ molar ratio of 20 to ∞, using a C₂-C₁₂ alkane diamine of the formula H₂N—(CH₂)—NH₂, wherein n=2 to 10, as structure directing agent. The ZSM-22, as synthesized, tends to crystallize as agglomerates of elongated crystals having the size of about 0.5 to about 2.0 microns. Ball milling fractures these crystals into smaller size crystallites (about 0.1 microns).

EP 0 170 003 A1 discloses the synthesis of crystalline silicates (ISI-1) having a SiO₂:Al₂O₃ molar ratio of at least 10, in the absence of organic structure directing agent.

U.S. Pat. No. 9,266,744 discloses the synthesis of borosilicate TON-framework type molecular sieves in hydroxide media using 1,3-dimethylimidazolium iodide or hydroxide or 1,4-bis(N-methylimidazoloium)butane dibromide or dihydroxide as structure directing agent. The synthesis of pure-silica and borosilicate TON-framework type molecular sieves using 1,3-dimethylimidazolium hydroxide, in fluoride or hydroxide media, was also disclosed in R. H. Archer et al., Microporous and Mesoporous Materials, 130 (2010) 255-265.

J. E. Schmidt et al., Chemistry of Materials, 27, 3756-3762 (2015), discloses the use of 1,3-dimethylimidazolium hydroxide as structure directing agent in the synthesis of a pure-silica TON-framework type molecular sieve in fluoride media, and of an aluminosilicate TON-framework type molecular sieves having a SiO₂:Al₂O₂ molar ratio of 60 in hydroxide media.

Despite these advances, there remains a need for new molecular sieves (or zeolites) with desirable properties, in particular a small particle (or crystal) size. It is also important to identify more efficient methods for the synthesis of molecular sieves (or zeolites) to facilitate their preparation and/or to reduce associated costs, such as for the synthesis of molecular sieves of TON framework type, in particular the direct synthesis of aluminum-containing molecular sieves of TON framework type.

### SUMMARY

In a first aspect, the present disclosure relates to a method of making an aluminum-containing molecular sieve of TON framework type, comprising the following steps:
(a) preparing a synthesis mixture comprising water, a source of silica, a source of alumina, a structure directing agent (Q) comprising at least one cation selected from 1,3-dimethylimidazolium cations and 1-ethyl-3-methylimidazolium cations, a source of hydroxide ions (OH), a source of alkali and/or alkaline earth metal element (M), and a source of halide ions (W) different from fluoride ions;
(b) heating said synthesis mixture under crystallization conditions including a temperature of from 100 to 200° C. for a time sufficient to form crystals of said molecular sieve;
(c) recovering at least a portion of the molecular sieve from step (b); and
(d) optionally treating the molecular sieve recovered in step (c) to remove at least part of the structure directing agent (Q).

In a second aspect, the present disclosure relates to an aluminum-containing molecular sieve of TON framework type obtainable by (or obtained by) the method disclosed herein.

In a third aspect, the present disclosure relates to an aluminum-containing molecular sieve of TON framework type in the form of crystals having a maximal particle size and/or an average maximal particle size of less than 500 nm, wherein at least 50% of the molecular sieve crystals (as vol %) have an aspect ratio (L/D) of at most 3, as determined by scanning electron microscopy (SEM).

In a fourth aspect, the present disclosure relates to a process of converting an organic compound to a conversion product, which comprises contacting the organic compound with the molecular sieve according to any of the second or third aspect of the present disclosure.

These and other features and attributes of the present disclosure and their advantageous applications and/or uses will be apparent from the detailed description which follows. It will of course be appreciated that features described in relation to one aspect of the present invention may be incorporated into other aspects of the present invention. In particular, any two or more of the features described in this specification, including in this summary section, can be combined to form combinations of features not specifically described herein.

### DESCRIPTION OF THE DRAWINGS

FIG. 1 shows the powder XRD pattern of the as-synthesized product of Example 1.

FIG. 2 shows SEM images of the as-synthesized product of Example 1.

FIG. 3 shows the powder XRD pattern of the as-synthesized product of Example 3.

FIG. 4 shows SEM images of the as-synthesized product of Example 3.

FIG. 5 shows a SEM image of the as-synthesized product of Example 4.

FIG. 6 shows a SEM image of the as-synthesized product of Example 5.

FIG. 7 shows a SEM image of the as-synthesized product of Example 6.

FIG. 8 shows a SEM image of the as-synthesized product of Example 11.

FIG. 9 shows a SEM image of the as-synthesized product of Example 15.

FIG. 10 shows a SEM image of the as-synthesized product of Example 19.

FIG. 11 shows a SEM image of the as-synthesized product of Example 20.

FIG. 12 shows a SEM image of the as-synthesized product of Comparative Example 21.

FIG. 13 shows a SEM image of the as-synthesized product of Comparative Example 22.

FIG. 14 shows a SEM image of the as-synthesized product of Example 23.

FIG. 15 shows a SEM image of the as-synthesized product of Example 24.

FIGS. 16 and 17 show molar ratios and conditions used for the syntheses of Examples 1 to 20 and 21 to 24, respectively.

FIG. 18 shows a SEM image of the Reference material used in catalyst performance testing.

FIGS. 19 and 20 show the performance testing, in terms of conversion and selectivity, of catalysts based on Reference material and the materials of Examples 4 and 6 in the oligomerization of C₃-C₆ olefins.

FIG. 21 shows the distillate selectivity vs. olefin conversion of catalysts based on Reference material and the materials of Examples 4 and 6 in the oligomerization of C₃-C₆ olefins.

FIGS. 22 and 23 show the performance testing, in terms of conversion and selectivity, of catalysts based on Reference material and the materials of Examples 4 and 6 in the oligomerization of C₂-C₆ olefins.

FIG. 24 shows the distillate selectivity vs. olefin conversion of catalysts based on the Reference material and the materials of Examples 4 and 6 in the oligomerization of C₂-C₆ olefins.

### DETAILED DESCRIPTION

The present inventors have found that the use of imidazolium cations selected from 1,3-dimethylimidazolium cations and 1-ethyl-3-methylimidazolium (or 1-methyl-3-ethylimidazolium) cations as structure directing agents (SDAs) allows the preparation of aluminum-containing molecular sieves of TON framework type, in particular in the form of small crystals. The present disclosure therefore relates to methods of making aluminum-containing molecular sieves of TON framework type using said SDAs. The present disclosure also relates to small crystal forms of aluminum-containing molecular sieves of TON framework type, in particular aluminum-containing molecular sieves of TON framework type in the form of crystals having a maximal particle size (or longest dimension) and/or an average maximal particle size of less than 500 nm, wherein at least 50% of the molecular sieve crystals (as vol %) have an aspect ratio (L/D) of at most 3, and uses thereof.

As used herein, molecular sieves of TON framework type are as discussed in the “Atlas of Zeolite Framework Types,” Fifth edition, 2001. More specifically, molecular sieves of TON framework may comprise, e.g., ISI-1, KZ-2, Nu-10, Theta-1 and ZSM-22 zeolites. In the following, the expressions “zeolite” and “molecular sieve” can be used interchangeably.

More particularly, molecular sieves of TON framework type comprise a family of crystalline materials having one-dimensional 10-membered ring channels and are characterized, in their as-synthesized form (e.g., where the SDA has not been removed), by a X-ray diffraction pattern (XRD) as referenced in M. M. J. Treacy and J. B. Higgins, “Collection of Simulated XRD Powder Patterns for Zeolites”, Fifth Revised Edition, 2007, Published on behalf of the Structure Commission of the International Zeolite Association, for TON framework and more particularly for diethylamine Theta-1 (silica ZSM-22). It must be noted that minor variations in the diffraction pattern can result from the removal of at least part of the SDA (e.g., by calcination), from variations in the mole ratios of the framework species, such as variations in the silica to alumina ratio, as well as from variations in the size of the crystals. In particular, small crystals will lead to some peak broadening. Notwithstanding these minor perturbations, the basic crystal lattice structure remains unchanged.

In a first aspect, the present disclosure relates to a method of making an aluminum-containing molecular sieve of TON framework type, comprising the following steps:
(a) preparing a synthesis mixture comprising water, a source of silica, a source of alumina, a structure directing agent (Q) comprising at least one cation selected from 1,3-dimethylimidazolium cations and 1-ethyl-3-methylimidazolium cations, a source of hydroxide ions (OH), a source of alkali and/or alkaline earth metal element (M), and a source of halide ions (W) different from fluoride ions;
(b) heating said synthesis mixture under crystallization conditions including a temperature of from 100 to 200° C. for a time sufficient to form crystals of said molecular sieve;
(c) recovering at least a portion of the molecular sieve from step (b); and
(d) optionally treating the molecular sieve recovered in step (c) to remove at least part of the structure directing agent (Q).

The structure directing agent (Q) is selected from 1,3-dimethylimidazolium cations and 1-ethyl-3-methylimidazolium (or 1-methyl-3-ethylimidazolium) cations, i.e., from cations of following Formula I and/or II:

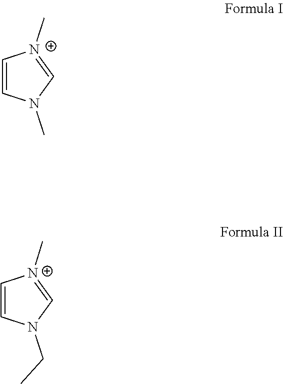

The structure directing agent (Q) may be present in any suitable form, for example as a halide, such as a fluoride, a chloride, an iodide or a bromide, as a hydroxide or as a nitrate, in particular in its hydroxide, iodide, chloride or bromide form, advantageously in its iodide, chloride or bromide form, e.g., in its chloride form. The structure directing agent (Q) may be present in the synthesis mixture in a Q/Si molar ratio of 0.01 to 1.0, such as from 0.02 to 0.7 or to 0.5, in particular from 0.03 to 0.3 or to less than 0.3, such as up to 0.2 or up to less than 0.2, e.g., as low as 0.03, 0.05 to 0.1.

The synthesis mixture comprises at least one source of silica. Suitable sources of silica (e.g., silicon oxide sources) include silicates, e.g., tetraalkyl orthosilicates such as tetramethylorthosilicate (TMOS) and tetraethylorthosilicate (TEOS), fumed silica such as Aerosil® (available from Evonik), Cabosperse® (available from Cabot) and CabO-O-Sil® (available from DMS), precipitated silica such as Ultrasil® and Sipernat® 340 (available from Evonik) or Hi-Sil®, alkali metal silicates such as potassium silicate and sodium silicate, and aqueous colloidal suspensions of silica, for example, that sold by Grace under the tradename Ludox® or that sold by Evonik under the tradename Aerodisp®; preferably silicates, fumed silica, colloidal silica, precipitated silica, and alkali metal silicates, e.g., precipitated or fumed silica.

The synthesis mixture comprises at least one source of alumina. Suitable sources of alumina (e.g., aluminum oxide) include aluminum hydroxide, aluminum salts, especially water-soluble salts, such as aluminum sulfate, aluminum nitrate, alkali metal aluminates such as sodium or potassium aluminate, and aluminum alkoxides such as aluminum isopropoxide, as well as hydrated aluminum oxides, such as boehmite, gibbsite, and pseudoboehmite, and mixtures thereof. Other aluminum sources include, but are not limited to, other water-soluble aluminum salts, or aluminum metal, such as aluminum in the form of chips. Especially suitable sources of alumina are aluminum hydroxide and water-soluble salts, such as aluminum sulfate, aluminum nitrate, and alkali metal aluminates, in particular sodium aluminate and potassium aluminate.

Alternatively or in addition to previously mentioned sources of Si and Al, sources containing both Si and Al elements can also be used. Examples of suitable sources containing both Si and Al elements include amorphous silica-alumina gels or dried silica alumina powders, silica aluminas, clays, such as kaolin, metakaolin, and zeolites, in particular aluminosilicates such as synthetic faujasite and ultrastable faujasite, for instance Y-Type Zeolite, Ultrastable Y (USY), beta or other large to medium pore molecular sieves or zeolites.

The synthesis mixture may advantageously have a silica to alumina (SiO₂/Al₂O₃ or Si/Al₂) molar ratio of from 25 to 300, such as from 30 or from more than 30 to 250, e.g., from 35 to 200.

Optionally, the synthesis mixture may comprise sources of other trivalent elements, such as boron. In embodiments where boron is present, suitable boron sources may include boric acid and borate salts such as sodium tetraborate or borax and potassium tetraborate. In embodiments where boron is present, the molecular sieve is an aluminoborosilicate, and the SiO₂/B₂O₃ (or Si/B₂) molar ratio may vary from 10 to infinity, such as from 10 to 500, or from 10 to 250. In embodiments where boron is present, the molecular sieve may have a SiO₂/Y₂O₃ (or Si/Y₂), where Y is a trivalent element comprising Al and B, of from 25 to 300, such as 30 or from more than 30 to 250. Alternatively, the synthesis mixture may be substantially free from a source of boron. In embodiments where boron is absent, the molecular sieve is an aluminosilicate.

The synthesis mixture comprises at least one source of hydroxide ions (OH). For example, hydroxide ions can be present as a counter ion of the structure directing agent (Q) or by the use of aluminum hydroxide or sodium aluminate as a source of Al. Suitable sources of hydroxide ions can also be selected from the group consisting of alkali metal hydroxides, alkaline earth metal hydroxides, ammonium hydroxide, and mixtures thereof, such as from sodium hydroxide, potassium hydroxide, lithium hydroxide, rubidium hydroxide, cesium hydroxide, calcium hydroxide, magnesium hydroxide, ammonium hydroxide, and mixtures thereof, more often sodium hydroxide, potassium hydroxide, lithium hydroxide, ammonium hydroxide, and mixtures thereof, most often sodium hydroxide and/or potassium hydroxide. The synthesis mixture may comprise the hydroxide ions source in a OH/Si molar ratio of from 0.01 to 1.0, such as from 0.05 to 0.8, or from 0.08 to 0.7, for instance from 0.1 to 0.5, e.g., from 0.1 to 0.3, or from 0.15 to 0.25, or 0.2.

The synthesis mixture comprises one or more sources of alkali or alkaline earth metal cation (M). M is preferably selected from the group consisting of sodium, potassium, lithium, rubidium, cesium, calcium, magnesium, strontium, barium, and mixtures thereof, preferably sodium and/or potassium, e.g., sodium and potassium, or sodium. In embodiments where M is sodium, suitable sodium sources include sodium hydroxide, sodium aluminate, sodium silicate, or sodium salts such as NaCl, NaBr or sodium nitrate. In embodiments where M is potassium, suitable potassium sources include potassium hydroxide, potassium aluminate, potassium silicate, a potassium salt such as KCl or KBr or potassium nitrate. In embodiments where M is lithium, suitable lithium sources include lithium hydroxide or lithium salts such as LiCl, LiBr, LiI, lithium nitrate, or lithium sulfate. In embodiments where M is rubidium, suitable rubidium sources include rubidium hydroxide or rubidium salts such as RbCl, RbBr, RbI, or rubidium nitrate. In embodiments where M is cesium, suitable cesium sources include cesium hydroxide. In embodiments where M is calcium, suitable calcium sources include calcium hydroxide. In embodiments where M is magnesium, suitable magnesium sources include magnesium hydroxide. In embodiments where M is strontium, suitable strontium sources include strontium hydroxide. In embodiments where M is barium, suitable barium sources include barium hydroxide. The alkali or alkaline earth metal cation M may also be present in the one or more sources of aluminum, such as sodium aluminate, potassium aluminate, or of any optional trivalent element, such as sodium tetraborate, potassium tetraborate and/or in the one or more sources of silica, such as potassium silicate and/or sodium silicate. The source of alkali or alkaline earth metal cation (M) is advantageously soluble in water. The synthesis mixture may comprise the alkali or alkaline earth metal cation (M) source in a M/Si molar ratio of from 0.01 to 1.0, such as from 0.05 to 0.8, advantageous at least 0.08, such as from 0.08 to 0.7, for instance from 0.1 to 0.5, e.g., from 0.1 to 0.3, or from 0.15 to 0.25, or 0.2.

The synthesis mixture comprises at least one source of halide ions (W), different from fluoride ions, which may be selected from the group consisting of chloride, bromide or iodide. The source of halide ions (W) may be any compound capable of releasing halide ions in the molecular sieve synthesis mixture. For instance, halide ions can be present as a counter ion of the structure directing agent (Q). Non-limiting examples of sources of halide ions include hydrogen chloride, ammonium chloride, hydrogen bromide, ammonium bromide, hydrogen iodide, and ammonium iodide; salts containing one or several halide ions, such as metal halides, preferably where the metal is an alkali or alkaline earth metal such as sodium, potassium, calcium, magnesium, strontium or barium; or tetraalkylammonium halides such as tetramethylammonium halides or tetraethylammonium halides. Small amounts of halide ions (W) may also be present as impurities, for instance in the optional source of alkali or alkaline earth metal cation (M). The halide ions (W) may advantageously be present in a W/Si molar ratio of 0.01 to 1.0, such as from 0.02 to 0.7 or to 0.5, or 0.03 to 0.3 or to 0.2, in particular 0.03 to less than 0.2, e.g., 0.03 or 0.05 to 0.1.

The synthesis mixture is preferably substantially free of fluoride ions (F). This means that no source of fluoride ions is added to the synthesis mixture in any substantial amount, e.g., the synthesis mixture has a F/Si molar ratio of less than 0.05, in particular less than 0.01 or even less than 0.005, such as 0. Said fluoride ions (F), if present, may originate from any compound capable of releasing fluoride ions in the molecular sieve synthesis mixture, such as hydrogen fluoride (HF); salts containing one or several fluoride ions, such as metal fluoride, preferably where the metal is an alkali or alkaline earth metal such as sodium, potassium, calcium, magnesium, strontium or barium, or a metal such as aluminum (AlF₃, Al₂F₆) or tin (SnF₂); ammonium fluoride (NH₄F); and ammonium bifluoride (NH₄HF₂). Small amounts of fluoride ions (F) may also be present as impurities, for instance in the optional source of alkali or alkaline earth metal cation (M).

The synthesis may be performed with or without added nucleating seeds. If nucleating seeds are added to the synthesis mixture, the seeds may be of the same or of a different framework type than TON, but preferably molecular sieves of TON framework type, such as ISI-1, KZ-2, Nu-10, Theta-1 and/or ZSM-22 seeds. Seeds may suitably be present in an amount from about 0.01 ppm by weight to about 20,000 ppm by weight, based on the synthesis mixture, such as from about 100 ppm by weight to about 10,000 ppm by weight of the synthesis mixture.

The synthesis mixture typically comprises water in a H₂O/Si molar ratio of from 1 to 100, such as 5 to 80 or 5 to 70, e.g., 10 or 15 to 50. Depending on the nature of the components in the base mixture, the amount of solvent (e.g., water from the hydroxide solution, and optionally methanol and ethanol from the hydrolysis of silica sources) of the base mixture may be removed such that a desired solvent to Si molar ratio is achieved for the synthesis mixture. Suitable methods for reducing the solvent content may include evaporation under a static or flowing atmosphere such as ambient air, dry nitrogen, dry air, or by spray drying or freeze drying. Water may also be added to the resulting mixture to achieve the desired H₂O/Si molar ratio when too much water is removed during the solvent removal process. In some examples, water removal is not necessary when the preparation have sufficient H₂O/Si molar ratio.

Carbon in the form of CH₂ may be present in the various sources of components used to prepare the synthesis mixture of the present disclosure, e.g., silica source or alumina source, and incorporated into the resulting molecular sieve framework as bridging atoms. Nitrogen atoms may be incorporated into the framework of the molecular sieve material as bridging atoms after the SDA has been removed.

In one or more aspects, the synthesis mixture after solvent adjustment (e.g., where the desired water to silica ratio is achieved) may be mixed by a mechanical process such as stirring or high shear blending to assure suitable homogenization of the base mixture, for example, using dual asymmetric centrifugal mixing (e.g., a FlackTek speedmixer) with a mixing speed of 1000 to 3000 rpm (e.g., 2000 rpm).

The synthesis mixture is then subject to crystallization conditions suitable for the molecular sieve material to form. Crystallization of the molecular sieve material may be carried out under static or stirred conditions in a suitable reactor vessel, such as for example, Teflon® lined or stainless-steel autoclaves placed in a convection oven maintained at an appropriate temperature.

The crystallization in step (b) of the method is typically carried out at a temperature of 100° C. to 200° C., such as 120° C. or 140° C. to less than 175° C., preferably up to 170° C. or less, e.g., 150° C. to 160° C., for a time sufficient for crystallization to occur at the temperature used. For instance, at higher temperatures, the crystallization time may be reduced. For instance, the crystallization conditions in step (b) of the method may include heating for a period of from 1 to 100 days, such as from 1 to 50 days, for example from 1 to 30 days. Advantageously, crystallization conditions may include a period of time as short as less than 10 days, or even less than 7 days, in particular 1 to 5 days, e.g., 2 to 3 days. The crystallization time can be established by methods known in the art such as by sampling the synthesis mixture at various times and determining the yield and X-ray crystallinity of precipitated solid. Unless indicated otherwise herein, the temperature measured is the temperature of the surrounding environment of the material being heated, for example the temperature of the atmosphere in which the material is heated.

Typically, the molecular sieve is formed in solution and can be recovered by standard means, such as by centrifugation or filtration. The separated molecular sieve can also be washed, recovered by centrifugation or filtration and dried.

The molecular sieve of the present disclosure, when employed either as an adsorbent or as a catalyst in an organic compound conversion process may be dehydrated (e.g., dried) at least partially. This can be done by heating to a temperature in the range of 80° C. to 500° C., such as 90° C. to 370° C. in an atmosphere such as air, nitrogen, etc., and at atmospheric, subatmospheric or superatmospheric pressures for between 30 minutes and 48 hours. Dehydration may also be performed at room temperature merely by placing the molecular sieve in a vacuum, but a longer time is required to obtain a sufficient amount of dehydration.

As a result of the crystallization process, the recovered product contains within its pores at least a portion of the structure directing agent used in the synthesis. The as-synthesized molecular sieve recovered from step (c) may thus be subjected to thermal treatment or other treatment to remove part or all of the SDA incorporated into its pores during the synthesis. Thermal treatment (e.g., calcination) of the as-synthesized molecular sieve typically exposes the materials to high temperatures sufficient to remove part or all of the SDA, in an atmosphere selected from air, nitrogen, ozone or a mixture thereof in a furnace. While subatmospheric pressure may be employed for the thermal treatment, atmospheric pressure is desired for reasons of convenience. The thermal treatment may be performed at a temperature up to 925° C. e.g., 300° C. to 700° C. or 400 to 600° C. The temperature measured is the temperature of the surrounding environment of the sample. The thermal treatment (e.g., calcination) may be carried out in a box furnace in dry air, which has been exposed to a drying tube containing drying agents that remove water from the air. The material is usually calcined for at least 1 minute and generally no longer than 1 or at most a few days. The heating may first be carried out under a nitrogen atmosphere and then the atmosphere may be switched to air and/or ozone.

The molecular sieve may also be subjected to an ion-exchange treatment, for example, with aqueous ammonium salts, such as ammonium nitrates, ammonium chlorides, and ammonium acetates, in order to remove remaining alkali metal cations and/or alkaline earth metal cations and to replace them with protons thereby producing the acid form of the molecular sieve. To the extent desired, the original cations of the as-synthesized material, such as alkali metal cations, can be replaced by ion exchange with other cations. Preferred replacing cations can include hydrogen ions, hydrogen precursor, e.g., ammonium ions and mixtures thereof. The ion exchange step may take place after the as-made molecular sieve is dried. The ion-exchange step may take place either before or after a calcination step.

The molecular sieve may also be subjected to other treatments such as steaming and/or washing with solvent. Such treatments are well-known to the skilled person and are carried out in order to modify the properties of the molecular sieve as desired.

The method of the first aspect of the present disclosure is especially advantageous in that it allows the direct preparation of aluminosilicate molecular sieves of TON framework type, with a wide range of silica to alumina ratio (such as from 25 to 300), under hydroxide media. There is thus no need for subsequent ion-exchange of boron-containing materials into aluminum-containing materials. Also, the synthesis does not require hydrofluoric acid.

The method of the present disclosure is also advantageous in that it allows the preparation of small crystal forms of aluminum-containing molecular sieve of TON framework type, such as crystals having a maximal particle size and/or an average maximal particle size of less than 500 nm, or even less than 300 nm. Small crystal size is advantageous in catalysis and adsorption applications as it allows for faster diffusion rates and higher surface areas. Without wishing to be bound by theory, it is believed that the small crystal forms of molecular sieves according to the present disclosure would improve selectivity and less secondary cracking activity when used as or in catalysts.

The method of the present disclosure is also advantageous in that it produces molecular sieves of TON framework type with a controlled morphology, in particular in the form of crystals having an aspect ratio (L/D) lower than 10, advantageously wherein at least 50% of the molecular sieve crystals (as vol %) have an aspect ratio (L/D) of at most 3. Materials having an aspect ratio (L/D) lower than 10 are advantageous over materials in the form of needles, sticks, laths, rods or fibrous crystals having an aspect ratio (L/D) higher than 10 that have produced concern as to the health effects of inhalation over a long period of time. Also, needle-like morphologies (or more generally morphologies with aspect ratios >10) has never been the most favorable morphology for catalytic applications due to the low diffusion rate via the long needles, which usually led to coking and side reactions (by products).

The method of the present disclosure is also advantageously conducted in the presence of small amounts of structure directing agent (Q), such as a Q/Si molar ratio of less than 0.3, or even less than 0.2, e.g., from 0.01 to less than 0.3, or from 0.02 to 0.2 or from 0.03 to less than 0.5, in particular from 0.03 to 0.1. This is advantageous as a lower amount of SDA results in a more cost-effective process. It is also advantageous because SDAs typically decompose at least partly in the synthesis mixture, so a lower amount of SDA will result in a lower amount of waste treatment. Without wishing to be bound by theory, it is believed that such small amounts of structure directing agent (Q), and/or a low SDA concentration relative to alkali and/or alkaline earth metal cations (M), such as a Q/M molar ratio of less than 2, in particular less than 1, and even more particularly of less than 0.8, such as from 0.05 to 0.7, or from 0.1 to 0.6, e.g., from 0.15 to 0.5, contributes to the preparation of TON crystals having a smaller particle size and/or a shorter aspect ratio (L/D).

In a second aspect, the present disclosure relates to an aluminum-containing molecular sieve of TON framework type, e.g., ISI-1, KZ-2, Nu-10, Theta-1 and/or ZSM-22, obtainable by (or obtained by) the method disclosed herein.

In its as-synthesized form (e.g., where the SDA has not been removed), the molecular sieve material of the present disclosure, in particular the molecular sieve of the second aspect of the present disclosure, comprises at least one cation selected from 1,3-dimethylimidazolium cations and 1-ethyl-3-methylimidazolium (or 1-methyl-3-ethylimidazolium) cations within its pore structure. Said molecular sieve may be represented, by the molecular formula of Formula III:

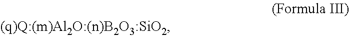

wherein 0<q≤1.0, 0.003≤m≤0.03, 0≤n≤0.1, and Q is at least one cation selected from 1,3-dimethylimidazolium cations and 1-ethyl-3-methylimidazolium (or 1-methyl-3-ethylimidazolium) cations. The oxygen atoms in Formula III may be replaced by carbon atoms (e.g., in the form of CH₂), which can come from sources of the components used to prepare the as-made molecular sieve. The oxygen atoms in Formula III can also be replaced by nitrogen atoms, e.g., after the SDA has been removed. Formula III can represent the framework of a typical molecular sieve material having structure directing agent (Q) within its pore structure and is not meant to be the sole representation of such material. The molecular sieve may contain impurities which are not accounted for in Formula III. Further, Formula III does not include the protons and charge compensating ions that may be present in the molecular sieve material.

The variable m represents the molar ratio relationship of Al₂O₃ to SiO₂ in Formula III. For example, when m is 0.01, the molar ratio of SiO₂ to Al₂O₃ is 100 and the Si/Al molar ratio is 50. m may vary from 0.003 to 0.04 or to 0.03, in particular from 0.004 to less than 0.03, e.g., from 0.005 to 0.028. The molar ratio of SiO₂ to Al₂O₃ may be from 25 or from 30 to 300, in particular from more than 30 to 250, e.g., 35 to 200.

The variable n represents the molar ratio relationship of B₂O₃ to SiO₂ in Formula III. For example, when n is 0.01, the molar ratio of SiO₂ to B₂O₃ is 100 and the Si/B molar ratio is 50. n may vary from 0 to 0.1, in particular from 0.002 to 0.1, e.g., from 0 to 0.02. The molar ratio of SiO₂ to B₂O₃ may be from 10 to infinity, such as (when B is present) from 10 to 500, e.g., from 10 to 250.

The variable q represents the molar relationship of Q to SiO₂ in Formula III. For example, when q is 0.1, the Q/Si molar ratio is 0.1. The molar ratio of Q to SiO₂ may be from higher than 0 to 1.0, such as from 0.01 to 1.0, or from 0.02 to 0.7 or to 0.5, such as from 0.03 to 0.3 or to less than 0.3, such as to less than 0.2, e.g., 0.03 to 0.1.

In its calcined form (e.g., where at least part of the SDA has been removed via thermal treatment or other treatment), the molecular sieve material of the second aspect of the present disclosure may be represented, by the molecular formula of Formula IV:

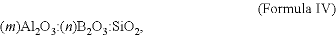

wherein 0.003≤m≤0.03 and 0≤n≤0.1. The oxygen atoms in Formula IV may be replaced by carbon atoms (e.g., in the form of CH₂), which can come from sources of the components used to prepare the as-made molecular sieve. The oxygen atoms in Formula IV can also be replaced by nitrogen atoms, e.g., after the SDA has been removed. Formula IV can represent the framework of a typical molecular sieve as defined in the present disclosure, in its calcined form, and is not meant to be the sole representation of said molecular sieve. Said molecular sieve, in its calcined form, may contain SDA and/or impurities after appropriate treatments to remove the SDA and impurities, which are not accounted for in Formula IV. Further, Formula IV does not include the protons and charge compensating ions that may be present in the calcined molecular sieve. The variable m represents the molar ratio relationship of Al₂O₃ to SiO₂ in Formula IV. The variable n represents the molar ratio relationship of B₂O₃ to SiO₂ in Formula IV. The values for variables m and n in Formula IV are the same as those described herein for Formula III.

In a further or another embodiment, advantageously, at least a portion of the molecular sieve of the present disclosure, whether in as-synthesized or calcined form, may be in the form of small crystals having a maximal particle size of less than 500 nm, in particular less than 300 nm, such as 250 nm or less, or 200 nm or less, or even 100 nm or less, e.g., from 50 to 200 nm, or from 100 to 200 nm, or from 50 to 100 nm. By “at least a portion” is meant at least about 50% of the molecular sieve particles (or crystals), such as at least 60%, at least 75%, or at least 85%. The maximal particle size of a particle (or crystal or crystallite) corresponds to the longest dimension (or length) of the particle. The maximal particle size of the crystals as well as the percentage (as vol %) of particles having a certain maximal size can be determined by image analysis, for example, of scanning electron microscopy (SEM) micrographs, e.g., using ImageJ software.

In a still further or another embodiment, the molecular sieve of the present disclosure, whether in as-synthesized or calcined form, may be in the form of crystals having an average maximal particle size of less than 500 nm, particularly less than 300 nm, such as 250 nm or less, or 200 nm or less, or even 100 nm or less, e.g., from 50 to less than 200 nm, or from 100 to less than 200 nm, or from 50 to less than 100 nm. The average maximal particle size can be defined as the average (or arithmetic mean) of the maximal particle size (or longest dimension or length) of particles (or crystals or crystallites) measured from randomly selected SEM micrographs from which were selected at least one hundred particles.

In one or more further embodiments, at least a portion of the molecular sieve of the present disclosure, whether in as-synthesized or calcined form, may be in the form of crystals having an elongated morphology. By “at least a portion” is meant at least about 50% of the molecular sieve particles (or crystals), such as at least 60%, at least 75%, or at least 85%. By “elongated morphology” is meant crystals that are substantially in the form of elongated particles such as rods, cylinders, sticks or laths, for instance in the form of rice grains.

In one or more further embodiments, at least a portion of the molecular sieve of the present disclosure, whether in as-synthesized or calcined form, may be in the form of crystals that advantageously have an aspect ratio (L/D) of less than 10, such as 5 or less, in particular 3 or less, e.g., 1 to 3 or to less than 3, as determined by scanning electron microscopy (SEM). By “at least a portion” is meant at least about 50% of the molecular sieve particles (or crystals), such as at least 60%, at least 75%, or at least 85%. The aspect ratio (L/D) of a crystal represents the ratio of the crystal length (L) (or the maximal particle size of the crystal), corresponding to the longest dimension of the crystal, to its diameter or width or thickness (D), corresponding to the smallest dimension of the crystal measured at the middle of the long dimension, perpendicular to said long dimension (shortest dimension orthogonal to the long dimension). The aspect ratio (L/D) of the particles (or crystals) as well as the percentage (as vol %) of particles (or crystals) having a certain aspect ratio can be determined by image analysis, for example, of scanning electron microscopy (SEM) micrographs, e.g., using ImageJ software.

In a third aspect, the present disclosure relates to aluminum-containing molecular sieves of TON framework type, whether in as-synthesized or calcined form, in the form of crystals having a maximal particle size and/or an average maximal particle size of less than 500 nm, wherein at least 50% of the molecular sieve crystals (as vol %) have an aspect ratio (L/D) of at most 3. The above-referenced values of maximal particle size and/or an average maximal particle size and of aspect ratio can be combined independently, so that, for instance, the present disclosure relates to aluminum-containing molecular sieves of TON framework type in the form of crystals having a maximal particle size and/or an average maximal particle size of less than 500 nm, in particular less than 300 nm, such as 250 nm or less, or 200 nm or less, e.g., from 50 to less than 200 nm, or from 100 to less than 200 nm, or from 50 to less than 100 nm, and an aspect ratio (L/D) of at most 3, such as 1 to 3, e.g., 1 to less than 3, as determined by scanning electron microscopy (SEM).

In one or more further embodiments, the molecular sieve of the present disclosure, whether in as-synthesized or calcined form, may have a silica to alumina (SiO₂/Al₂O₃ or Si/Al₂) molar ratio of from 25 to 300, such as from 30 or from more than 30 to 250, e.g., from 35 to 200, as measured by X-ray fluorescence (XRF).

In one or more further embodiments, the molecular sieve of the present disclosure, in its calcined form, may have a BET surface area (S_{BET}) of from 120 to 400 m²/g, such as from 180 to 300 m²/g; e.g., from 200 to 275 m²/g; a micropore surface area (S_{micro}) of from 75 to 300 m²/g, such as from 85 to 250 m²/g, e.g., from 90 to 200 m²/g; and/or an external surface area (Sₑ,t) of from 25 to 250 m²/g, such as from 30 to 200 m²/g, e.g., from 35 to 180 m²/g; and/or a micropore volume (V_{micro}) of 0.02 to 0.2, or from 0.03 to 0.1, for example 0.04 to 0.08 cm³/g. In a further embodiment, the molecular sieve of the present disclosure, in its calcined form, may have an external surface area (Sₑ,t) of at least 50 m²/g, such as at least 75 m²/g, or at least 100 m²/g, e.g., from 125 to 250 m²/g, or from 150 to 200 m²/g.

In one or more further embodiments, the molecular sieve of the present disclosure may be ISI-1, KZ-2, Nu-10, Theta-1 and/or ZSM-22, e.g., ZSM-22.

The molecular sieve materials of the present disclosure, where part or all of the SDA has been removed, may be used as an adsorbent or as a catalyst or support for catalyst in a wide variety of hydrocarbon conversions, e.g., conversion of organic compounds to a converted product. In a fourth aspect, the present disclosure therefore relates to a process of converting an organic compound to a conversion product, which comprises contacting the organic compound with the molecular sieve of the present disclosure, in particular with the molecular sieve according to the second or third aspect of the present disclosure, or prepared according to the process of the first aspect of the present disclosure.

The molecular sieve materials of the present disclosure (where part or all of the SDA is removed) may be used as an adsorbent, such as for separating at least one component from a mixture of components in the vapor or liquid phase having differential sorption characteristics with respect to the material. Therefore, at least one component can be partially or substantially totally separated from a mixture of components having differential sorption characteristics with respect to the molecular sieve by contacting the mixture with said molecular sieve to selectively sorb the one component. For instance, in a process for selectively separating one or more desired components of a feedstock from remaining components of the feedstock, the feedstock may be contacted with a sorbent that comprises the molecular sieve of the present disclosure at effective sorption conditions, thereby forming a sorbed product and an effluent product. One or more of the desired components are recovered from either the sorbed product or the effluent product.

The molecular sieve of the present disclosure (where part or all of the SDA is removed) may also be used as a catalyst to catalyze a wide variety of organic compound conversion processes. Examples of chemical conversion processes, which are effectively catalyzed by the molecular sieve described herein, either alone or in combination with one or more other catalytically active substances including other crystalline catalysts, include those requiring a catalyst with acid activity. Examples of organic conversion processes, which may be catalyzed by the molecular sieve described herein, either alone or in combination with one or more other catalytically active substances, including other crystalline catalysts, include oligomerization, such as olefin oligomerization, cracking, hydrocracking, isomerization, polymerisation, reforming, hydrogenation, dehydrogenation, dewaxing, hydrodewaxing, adsorption, alkylation, transalkylation, dealkylation, hydrodecylization, disproportionation, dehydrocyclization, conversion of oxygenates to hydrocarbon, such as conversion of methanol to hydrocarbons, e.g., olefins and/or gasoline, applications, and combinations thereof. For instance, the molecular sieve of the present disclosure may be especially suitable in the oligomerization of olefin streams, more particularly of olefin feed streams containing olefins having from about 2 to about 12 carbon atoms (straight and/or branched), such as from about 2 to about 8 carbon atoms, in particular from 2 to 6 or from 3 to 6 carbon atoms. The oligomers which are the products of the oligomerization process are medium to heavy olefins (including trimer and heavier oligomer products) which are useful for fuels, i.e., distillate (i.e., jet and diesel) or gasoline or a gasoline blending stock, and chemicals. More details on olefin oligomerization are for instance given in US2004/0015028A1 or WO2005/118512 which are incorporated herewith in their entirety.

The conversion of hydrocarbon feeds can take place in any convenient mode, for example in fluidized bed, moving bed, or fixed bed reactors depending on the types of process desired.

The molecular sieve of the present disclosure may be formulated into product compositions by combination with other materials, such as binders and/or matrix materials that provide additional hardness to the finished product. These other materials can be inert or catalytically active materials.

For instance, it may be desirable to incorporate the molecular sieve of the present disclosure with another material that is resistant to the temperatures and other conditions employed during use. Such materials include synthetic or naturally occurring zeolites as well as inorganic materials such as clays, silica and/or metal oxides such as alumina and mixtures thereof. The metal oxides may be either naturally occurring or in the form of gelatinous precipitates or gels including mixtures of silica and metal oxides. Use of a resistant material in conjunction with the molecular sieve of the present disclosure, i.e., combined therewith or present during synthesis of the as-made molecular sieve, which crystal is active, tends to change the conversion and/or selectivity of the catalyst in certain organic conversion processes. Inactive resistant materials suitably serve as diluents to control the amount of conversion in a given process so that products can be obtained in an economic and orderly manner without employing other means for controlling the rate of reaction. These materials may be incorporated into naturally occurring clays, e.g., bentonite and kaolin, to improve the crush strength of the product under commercial operating conditions. Said inactive resistant materials, i.e., clays, oxides, etc., function as binders for the catalyst. A catalyst having good crush strength can be beneficial because in commercial use, it is desirable to prevent the catalyst from breaking down into powder-like materials.

Naturally occurring clays which may be used include the montmorillonite and kaolin family, which families include the subbentonites, and the kaolins commonly known as Dixie, McNamee, Georgia and Florida clays or others in which the main mineral constituent is halloysite, kaolinite, dickite, nacrite, or anauxite. Such clays can be used in the raw state as originally mined or after being subjected to calcination, acid treatment or chemical modification. Binders useful for compositing with the molecular sieve of the present disclosure also include inorganic oxides selected from silica, zirconia, titania, magnesia, beryllia, alumina, yttria, gallium oxide, zinc oxide and mixtures thereof.

In addition to the foregoing materials, the molecular sieve of the present disclosure may be composited with a porous matrix material such as silica-alumina, silica-magnesia, silica-zirconia, silica-thoria, silica-beryllia, silica-titania as well as ternary compositions such as silica-alumina-thoria, silica-alumina-zirconia, silica-alumina-magnesia and silica-magnesia-zirconia.

These binder materials are resistant to the temperatures and other conditions, e.g., mechanical attrition, which occur in various hydrocarbon separation processes. Thus, the molecular sieve of the present disclosure may be used in the form of an extrudate with a binder. They are typically bound by forming a pill, sphere, or extrudate. The extrudate is usually formed by extruding the molecular sieve, optionally in the presence of a binder, and drying and calcining the resulting extrudate. Further treatments such as steaming, and/or ion exchange may be carried out as required. The molecular sieve may optionally be bound with a binder having a surface area of at least 100 m²/g, for instance at least 200 m²/g, optionally at least 300 m²/g.

The relative proportions of molecular sieve and inorganic oxide matrix may vary widely, with the molecular sieve content ranging from about 1 to about 100 percent by weight and more usually, particularly when the composite is prepared in the form of extrudates, in the range of about 2 to about 95, optionally from about 20 to about 90 weight percent of the composite.

The molecular sieve of the present disclosure may also be used in intimate combination with a hydrogenating component such as tungsten, vanadium, molybdenum, rhenium, nickel, cobalt, chromium, manganese, or a noble metal such as platinum or palladium where a hydrogenation-dehydrogenation function is to be performed. Such hydrogenating components may be incorporated in the composition by way of one or more of the following processes: cocrystallization; exchanged into the composition to the extent a Group IIIA element, e.g., aluminum, is in the structure; or intimately physically admixed therewith. Such components can also be impregnated in or onto the molecular sieve, for example, by treating the molecular sieve with a hydrogenating metal-containing ion. For instance, in the case of platinum, suitable platinum compounds for this purpose include chloroplatinic acid, platinous chloride and various compounds containing a platinum amine complex. Combinations of metals and methods for their introduction can also be used.

It will be understood by a person skilled in the art that the molecular sieve of the present disclosure may contain impurities, such as amorphous materials, unit cells having different topologies (e.g., quartz or molecular sieves of different framework type, that may or may not impact the performance of the resulting catalyst), and/or other impurities (e.g., heavy metals and/or organic hydrocarbons). Typical examples of molecular sieves of different framework type co-existing with the molecular sieve of the present disclosure are e.g., molecular sieves of MOR, MFI, or FER framework type, such as Mordenite, ZSM-5, or Ferrierite. The molecular sieve of the present disclosure is preferably substantially free of impurities. The term “substantially free of impurities” (or in the alternative “substantially pure”) used herein means the molecular sieve material contains a minor proportion (less than 50 wt %), preferably less than 20 wt %, more preferably less than 10 wt %, even more preferably less than 5 wt % and most preferably less than 1 wt % (e.g., less than 0.5 wt % or 0.1 wt %), of such impurities (or “non-TON” framework type material), which weight percent (wt %) values are based on the combined weight of impurities and pure molecular sieve. The amount of impurities can be appropriately determined by powder XRD, rotating electron diffraction, and/or SEM/TEM (e.g., different crystal morphologies).

The molecular sieve described herein is substantially crystalline. As used herein, the term “crystalline” refers to a crystalline solid form of a material, including, but not limited to, a single-component or multiple-component crystal form, e.g., including solvates, hydrates, and a co-crystal. Crystalline can mean having a regularly repeating and/or ordered arrangement of molecules, and possessing a distinguishable crystal lattice. For example, the molecular sieve can have different water or solvent content. The different crystalline lattices can be identified by solid state characterization methods such as by XRD (e.g., powder XRD). Other characterization methods known to a person of ordinary skill in the relevant art can further help identify the crystalline form as well as help determine stability and solvent/water content. As used herein, the term “substantially crystalline” means a majority (greater than 50 wt %) of the weight of a sample of a material described is crystalline and the remainder of the sample is a non-crystalline form. In one or more aspects, a substantially crystalline sample has at least 95% crystallinity (e.g., 5% of the non-crystalline form), at least 96% crystallinity (e.g., 4% of the non-crystalline form), at least 97% crystallinity (e.g., 3% of the non-crystalline form), at least 98% crystallinity (e.g., about 2% of the non-crystalline form), at least 99% crystallinity (e.g., 1% of the non-crystalline form), and 100% crystallinity (e.g., 0% of the non-crystalline form).

Aspects of the disclosure are described in greater detail by way of specific examples. The following examples are offered for illustrative purposes and are not intended to limit the disclosure in any manner. Those of skill in the relevant art will readily recognize a variety of parameters can be changed or modified to yield essentially the same results.

### EXAMPLES

The present invention is further illustrated below without limiting the scope thereto.

In these examples, the X-ray diffraction (XRD) patterns of the as-synthesized and calcined materials were recorded on a Bruker D8 Endeavor Automated X-Ray Powder Diffractometer in continuous mode using a Cu Kα radiation, Bragg-Bentano geometry with Vantec 500 detector, in the 20 range of 4 to 40 degrees. The interplanar spacings, d-spacings, were calculated in Angstrom units, and the relative intensities of the lines, I/Iₒ is the ratio of the peak intensity to that of the intensity of the strongest line, above background. The intensities are uncorrected for Lorentz and polarization effects. The location of the diffraction peaks in 2-theta, and the relative peak area intensities of the lines, II(o), where Iₒ is the intensity of the strongest line, above background, were determined with the MDI Jade peak search algorithm. It should be understood that diffraction data listed as single lines may consist of multiple overlapping lines which under certain conditions, such as differences in crystallographic changes, may appear as resolved or partially resolved lines. Typically, crystallographic changes can include minor changes in unit cell parameters and/or a change in crystal symmetry, without a change in the framework connectivity. These minor effects, including changes in relative intensities, can also occur as a result of differences in cation content, framework composition, nature and degree of pore filling, crystal size and shape, preferred orientation and thermal and/or hydrothermal history.

The scanning electron microscopy (SEM) images of the as-synthesized materials were obtained on a Hitachi S4800 Field Emission Scanning Electron Microscope. SEM images were used to aid assessment of product purity. The presence of obviously different crystal morphologies in a SEM image can be an indication of impurities in the form of other crystalline materials. Such an approximate analysis can be especially useful in identifying the presence of formation of relatively minor amounts of crystalline impurities which may not be identifiable on product XRD patterns.

The following measurements were conducted on samples that were ion-exchanged and calcined. For each sample subjected to ion-exchange and calcination, the procedure used was as follows: the as-prepared sample was calcined at 580° C. for 8 hours, ion-exchanged two times with a 1M ammonium nitrate solution and then calcined at 550° C. for 4 hours.

The overall BET surface area (S_{BET}) of the materials was determined by the BET method as described by S. Brunauer, P. H. Emmett and E. Teller, J Am. Chem. Soc., 1938, 60, 309, incorporated herein by reference, using nitrogen adsorption-desorption at liquid nitrogen temperature. The external surface area (Sₑₓₜ) of the material was obtained from the t-plot method, and the micropore surface area (S_{micro}) of the material was calculated by subtracting the external surface area (Sₑₓₜ) from the overall BET surface area (S_{BET}).

The micropore volume (V_{micro}) and total pore volume (Vₜₒₜ) of the materials can be determined using methods known in the relevant art. For example, the micropore and total pore volumes of the materials can be measured with nitrogen physisorption, and the data can be analyzed by the t-plot method described in Lippens, B. C. et al., “Studies on pore system in catalysts: V. The t method”, J. Catal., 4, 319 (1965), which describes micropore and total pore volume methods and is incorporated herein by reference.

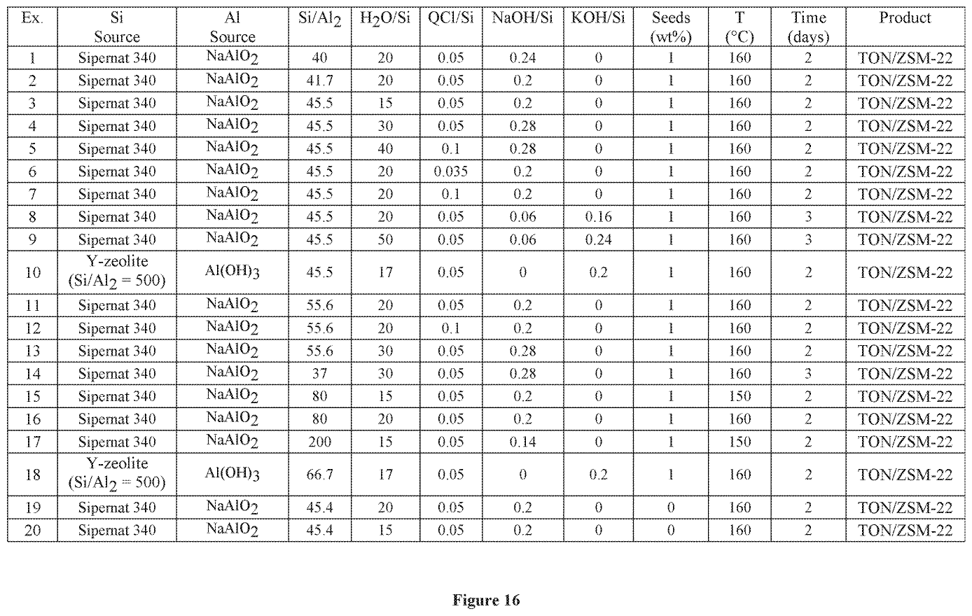

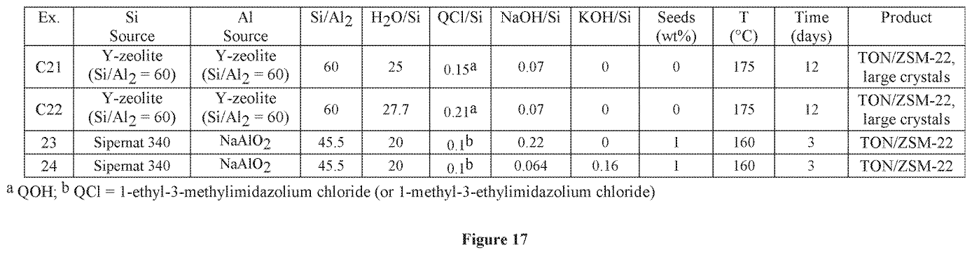

The molar ratios and conditions used for the syntheses of Examples 1 to 24, as well as the resulting products, are detailed below and summarized in FIGS. 16 and 17.

### Example 1: 1,3-dimethylimidazolium chloride

In a PTFE liner for a 23 mL Steel Parr autoclave, the following were mixed: 0.66 g of 1,3-dimethylimidazolium chloride solution (purchased from TCI, 20 wt %), 1.37 g of NaOH solution (10 wt %), 0.21 g of sodium aluminate (NaAlO₂, 23.5 wt % Al₂O₃, 19.4 wt % Na₂O), 5 g of deionized water, 100 mg of ZSM-22 seeds, and 1.19 g of precipitated silica (Sipernat® 340 available from Evonik), to produce a synthesis mixture having the following composition in terms of molar ratios:
20 H₂O: 1 SiO₂: 0.025 Al₂O₃: 0.05 QCl: 0.24 NaOH

The liner was then capped, sealed within a 23 mL Parr autoclave and placed within a spit inside of a convection oven. The reactor was heated at 160° C. for 2 days under tumbling conditions (about 30 rpm). The product was isolated by filtration, rinsed with deionized water, and dried.

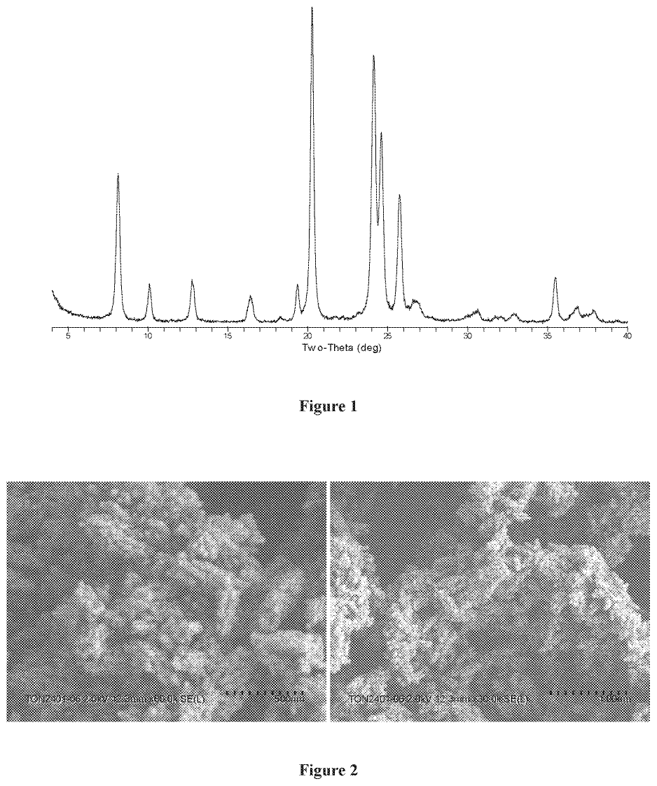

XRD analysis of the as-synthesized product, illustrated in FIG. 1, showed the material to have a powder XRD pattern of ZSM-22 or TON framework type. FIG. 2 shows SEM images of the as-synthesized product and indicates that the crystals have a uniform rice morphology (L/D<3) with an average particle length of from 50 to 100 nm.

### Example 2: 1,3-dimethylimidazolium chloride

This Example was conducted in similar conditions as Example 1 except for a higher silica to alumina molar ratio of 41.7 and NaOH in an amount NaOH/SiO₂ of 0.2, to produce a synthesis mixture having the following composition in terms of molar ratios:
20 H₂O: 1 SiO₂: 0.024 Al₂O₃: 0.05 QCl: 0.2 NaOH

After 2 days of heating at 160° C., a molecular sieve of TON framework type or ZSM-22 was obtained, as identified by its XRD pattern.

### Example 3: 1,3-dimethylimidazolium chloride

In a PTFE liner for a 23 mL steel Parr autoclave, the following were mixed: 0.88 g of 1,3-dimethylimidazolium chloride solution (purchased from TCI, 20 wt %), 1.49 g of NaOH solution (10 wt %), 0.25 g of sodium aluminate (NaAlO₂, 23.5 wt % Al₂O₃, 19.4 wt % Na₂O), 4.35 g of deionized water, 160 mg of ZSM-22 seeds, and 1.59 g of precipitated silica (Sipernat® 340 available from Evonik), to produce a synthesis mixture having the following composition in terms of molar ratios:
20 H₂O: 1 SiO₂: 0.022 Al₂O₃: 0.05 QCl: 0.20 NaOH

The liner was then capped, sealed within a 23 mL Parr autoclave, and placed within a spit inside of a convection oven. The reactor was heated at 160° C. for 2 days under tumbling conditions (about 30 rpm). The product was isolated by filtration, rinsed with deionized water, and dried.

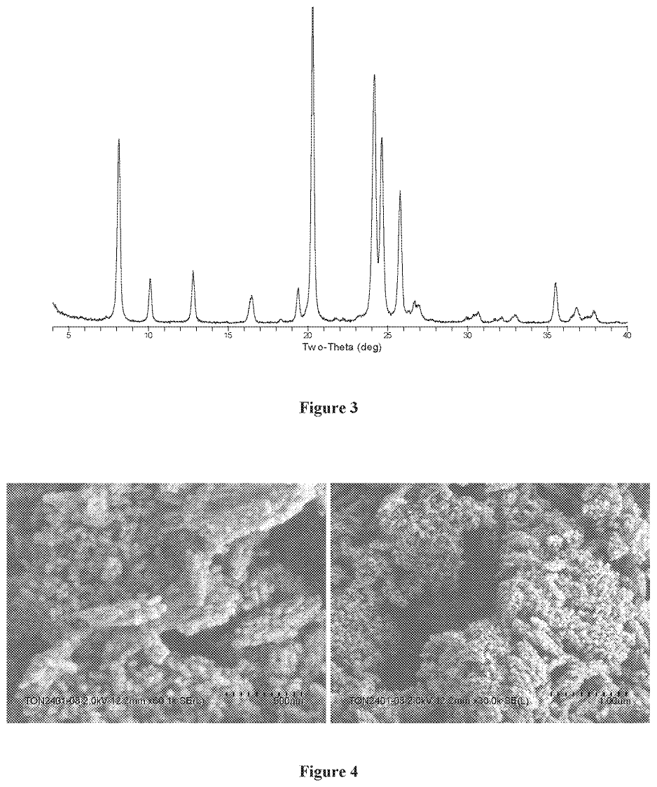

XRD analysis of the as-synthesized material, illustrated in FIG. 3, showed the material to have a powder XRD pattern of ZSM-22 or TON framework type. FIG. 4 shows SEM images of the as-synthesized material and indicates that the crystals have a uniform rice morphology (L/D<3) with an average particle length of about 100 nm.

### Example 4: 1,3-dimethylimidazolium chloride

In a PTFE liner for a 200 mL Steel Parr autoclave, the following were mixed: 8.24 g of 1,3-dimethylimidazolium chloride solution (purchased from TCI, 20 wt %), 21.9 g of NaOH solution (10 wt %), 100 g deionized water, 2.37 g of sodium aluminate (NaAlO₂, 23.5 wt % Al₂O₃, 19.4 wt % Na₂O), and 14.9 g of precipitated silica (Sipernat® 340 available from Evonik), with an addition of 0.15 g of ZSM-22 seeds, to produce a synthesis mixture having the following composition in terms of molar ratios:
30 H₂O: 1 SiO₂: 0.022 Al₂O₃: 0.05 QCl: 0.28 NaOH

The liner was then capped, sealed within a 200 mL Parr autoclave, and placed within a spit inside of a convection over. The reactor was heated at 160° C. for 2 days under tumbling conditions (about 30 rpm). The product was isolated by filtration, rinsed with deionized water, and dried.

XRD analysis of the as-synthesized material showed the material to have a powder XRD pattern of ZSM-22 or TON framework type. FIG. 5 shows a SEM image of the as-synthesized material and indicates that the crystals have a uniform rice morphology (L/D<3) with a particle length of from 50 to 100 nm. The material of Example 4 had a silica to alumina molar ratio of 38, as determined by XRF.

The micropore surface area (S_{micro}) of the calcined version of the material of Example 4 was 157 m²/g, its external surface area (Sₑₓt) was 46 m²/g, and its micropore volume (V_{micro}) was 0.062 cm³/g.

### Example 5: 1,3-dimethylimidazolium chloride

In a PTFE liner for a 200 mL Steel Parr autoclave, the following were mixed: 12.36 g of 1,3-dimethylimidazolium chloride solution (purchased from TCI, 20 wt %), 16.4 g of NaOH solution (10 wt %), 104 g of deionized water, 1.78 g of sodium aluminate (NaAlO₂, 23.5 wt % t Al₂O₂, 19.4 wt % Na₂O), and 11.2 g of precipitated silica (Sipernat® 340 available from Evonik), with an addition of 0.1 g of ZSM-22 seeds, to produce a synthesis mixture having the following composition in terms of molar ratios:
40 H₂O: 1 SiO₂: 0.022 Al₂O₂: 0.1 QCl: 0.28 NaOH

The liner was then capped, sealed within a 200 mL Parr autoclave, and placed within a spit inside of a convection over. The reactor was heated at 160° C. for 2 days under tumbling conditions (about 30 rpm). The product was isolated by filtration, rinsed with deionized water, and dried.

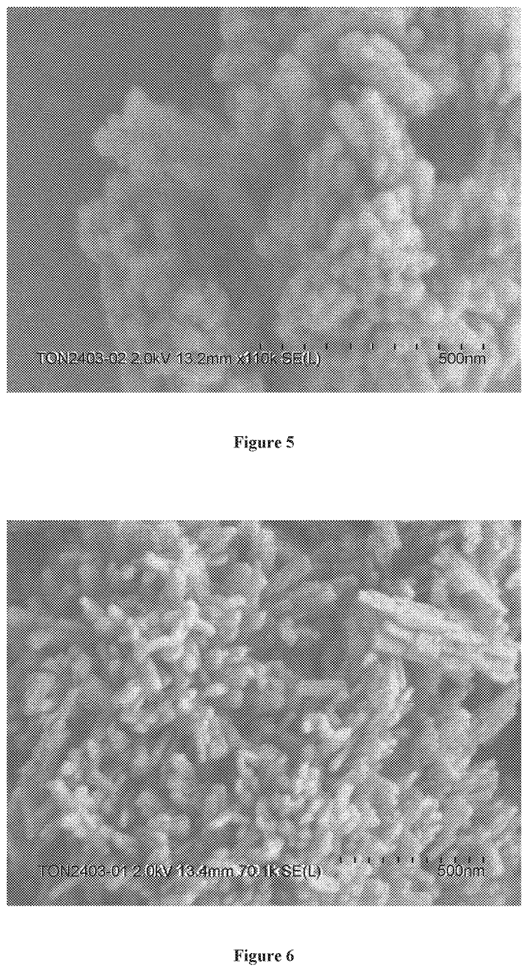

XRD analysis of the as-synthesized material showed the material to have a powder XRD pattern of ZSM-22 or TON framework type. FIG. 6 shows a SEM image of the as-synthesized material and indicates that the crystals have a uniform rice morphology (L/D≤3) with a particle length of from 100 to 200 nm.

### Example 6: 1,3-dimethylimidazolium chloride

In a PTFE liner for a 200 mL Steel Parr autoclave, the following were mixed: 8.1 g of 1,3-dimethylimidazolium chloride solution (purchased from TCI, 20 wt %), 19.5 g of NaOH solution (10 wt %), 91 g of deionized water, 3.32 g of sodium aluminate (NaAlO₂, 23.5 wt % Al₂O₃, 19.4 wt % Na₂O), and 20.9 g of precipitated silica (Sipernat® 340 available from Evonik), with an addition of 0.2 g of ZSM-22 seeds, to produce a synthesis mixture having the following composition in terms of molar ratios:
20 H₂O: 1 SiO₂: 0.022 Al₂O₃: 0.035 QCl: 0.2 NaOH

The liner was then capped, sealed within a 200 mL Parr autoclave, and placed within a spit inside of a convection over. The reactor was heated at 160° C. for 2 days under tumbling conditions (about 30 rpm). The product was isolated by filtration, rinsed with deionized water, and dried.

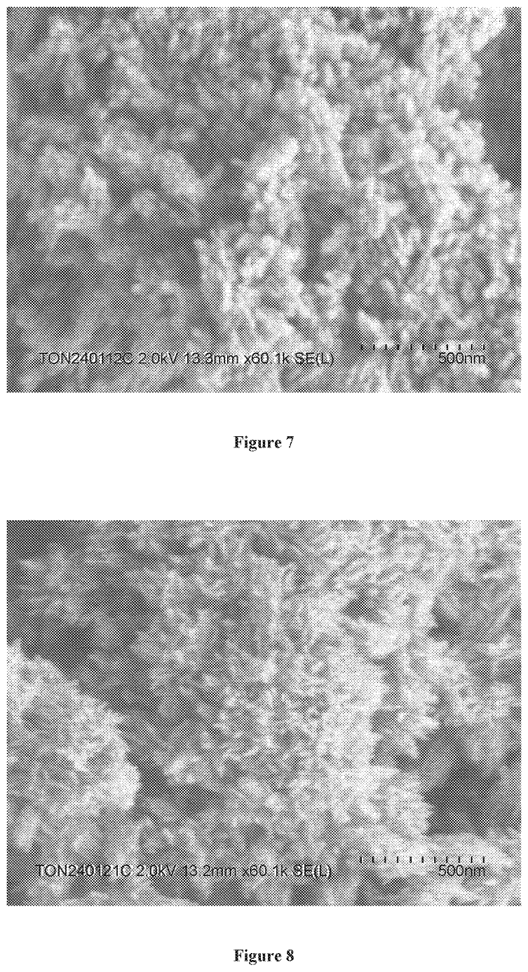

XRD analysis of the as-synthesized material showed the material to have a powder XRD pattern of ZSM-22 or TON framework type. FIG. 7 shows a SEM image of the as-synthesized product and indicates that the crystals have a uniform rice morphology (L/D≤3) with a particle length of from 50 to 100 nm. The material of Example 6 had a silica to alumina molar ratio of 40, as determined by XRF.

The micropore surface area (S_{micro}) of the calcined version of the TON material of Example 6 was 91 m²/g, its external surface area (Sₑₓₜ) was 178 m²/g, its micropore volume (V_{micro}) was 0.04 cm³/g.

### Example 7: 1,3-dimethylimidazolium chloride

This Example was conducted in similar conditions as Example 6 except for the structure directing agent in an amount QCl/SiO₂ of 0.1, to produce a synthesis mixture having the following composition in terms of molar ratios:
20 H₂O: 1 SiO₂: 0.022 Al₂O₃: 0.1 QCl: 0.2 NaOH

After 2 days of heating at 160° C., a molecular sieve of TON framework type or ZSM-22 was obtained, as identified by its XRD pattern.

### Example 8: 1,3-dimethylimidazolium chloride

This Example was conducted in similar conditions as Example 6 except for the structure directing agent in an amount QCl/SiO₂ of 0.05 and the addition of KOH, to produce a synthesis mixture having the following composition in terms of molar ratios:
20 H₂O: 1 SiO₂: 0.022 Al₂O₃: 0.05 QCl: 0.06 NaOH: 0.16 KOH

After 3 days of heating at 160° C., a molecular sieve of TON framework type or ZSM-22 was obtained, as identified by its XRD pattern.

### Example 9: 1,3-dimethylimidazolium chloride

This Example was conducted in similar conditions as Example 8 except higher amounts of water and KOH, to produce a synthesis mixture having the following composition in terms of molar ratios:
50 H₂O: 1 SiO₂: 0.022 Al₂O₃: 0.05 QCl: 0.06 NaOH: 0.25 KOH

After 3 days of heating at 160° C., a molecular sieve of TON framework type or ZSM-22 was obtained, as identified by its XRD pattern.

### Example 10: 1,3-dimethylimidazolium chloride

In a PTFE liner for a 23 mL Steel Parr autoclave, the following were mixed: 0.87 g of 1,3-dimethylimidazolium chloride solution (purchased from TCI, 20 wt %), 1.46 g of KOH solution (20 wt %), 6.11 g of deionized water, 0.077 g of Al(OH)₃ (76 wt % Al₂O₃, Alfa), and 1.57 g of Y-type zeolite with a Si/Al₂ molar ratio of 500 (available from Tosoh as HSZ-390HUA), with an addition of 0.1 g of ZSM-22 seeds, to produce a synthesis mixture having the following composition in terms of molar ratios:
17 H₂O: 1 SiO₂: 0.022 Al₂O₃: 0.05 QCl: 0.2 KOH

The liner was then capped, sealed within a 23 mL Parr autoclave, and placed within a spit inside of a convection over. The reactor was heated at 160° C. for 2 days under tumbling conditions (about 30 rpm). The product was isolated by filtration, rinsed with deionized water, and dried.

XRD analysis of the as-synthesized material showed the material to have a powder XRD pattern of ZSM-22 or TON framework type.

### Example 11: 1,3-dimethylimidazolium chloride

In a PTFE liner for a 200 mL Steel Parr autoclave, the following were mixed: 11.53 g of 1,3-dimethylimidazolium chloride solution (purchased from TCI, 20 wt %), 21 g of NaOH solution (10 wt %), 88.8 g of deionized water, 2.72 g of sodium aluminate (NaAlO₂, 23.5 wt % Al₂O₃, 19.4 wt % Na₂O), and 20.9 g of precipitated silica (Sipernat® 340 available from Evonik), with an addition of 0.2 g of ZSM-22 seeds, to produce a synthesis mixture having the following composition in terms of molar ratios:
20 H₂O: 1 SiO₂: 0.018 Al₂O₃: 0.05 QCl: 0.2 NaOH

The liner was then capped, sealed within a 200 mL Parr autoclave, and placed within a spit inside of a convection over. The reactor was heated at 160° C. for 2 days under tumbling conditions (about 30 rpm). The product was isolated by filtration, rinsed with deionized water, and dried.

XRD analysis of the as-synthesized material showed the material to have a powder XRD pattern of ZSM-22 or TON framework type. FIG. 8 shows a SEM image of the as-synthesized product and indicates that the crystals have a uniform rice morphology (L/D about 3) with a particle length of about 200 nm.

The micropore surface area (S_{micro}) of the calcined version of the TON material of Example 11 was 187 m²/g, its external surface area (Sₑₓₜ) was 36 m²/g, its micropore volume (V_{micro}) was 0.072 cm³/g.

### Example 12: 1,3-dimethylimidazolium chloride

This Example was conducted in similar conditions as Example 11 except for the structure directing agent in an amount QCl/SiO₂ of 0.1, to produce a synthesis mixture having the following composition in terms of molar ratios:
20 H₂O: 1 SiO₂: 0.018 Al₂O₃: 0.1 QCl: 0.2 NaOH

After 2 days of heating at 160° C., a molecular sieve of TON framework type or ZSM-22 was obtained, as identified by its XRD pattern.

### Example 13: 1,3-dimethylimidazolium chloride

This Example was conducted in similar conditions as Example 11 except for NaOH in an amount NaOH/SiO₂ of 0.28, to produce a synthesis mixture having the following composition in terms of molar ratios:
20 H₂O: 1 SiO₂: 0.018 Al₂O₃: 0.05 QCl: 0.28 NaOH

After 2 days of heating at 160° C., a molecular sieve of TON framework type or ZSM-22 was obtained, as identified by its XRD pattern.

### Example 14: 1,3-dimethylimidazolium chloride

This Example was conducted in similar conditions as Example 13 except for silica to alumina molar ration of 37, to produce a synthesis mixture having the following composition in terms of molar ratios:
20 H₂O: 1 SiO₂: 0.027 Al₂O₃: 0.05 QCl: 0.28 NaOH

After 2 days of heating at 160° C., a molecular sieve of TON framework type or ZSM-22 was obtained, as identified by its XRD pattern.

### Example 15: 1,3-dimethylimidazolium chloride

In a PTFE liner for a 200 mL Steel Parr autoclave, the following were mixed: 2.86 g of 1,3-dimethylimidazolium chloride solution (purchased from TCI, 100 wt %), 5.7 g of NaOH solution (50 wt %), 111 g of deionized water, 2.34 g of sodium aluminate (NaAlO₂, 23.5 wt % Al₂O₃, 19.4 wt % Na₂O), and 25.9 g of precipitated silica (Sipernat® 340 available from Evonik), with an addition of 0.3 g of ZSM-22 seeds, to produce a synthesis mixture having the following composition in terms of molar ratios:
15 H₂O: 1 SiO₂: 0.0125 Al₂O₃: 0.05 QCl: 0.2 NaOH

The liner was then capped, sealed within a 200 mL Parr autoclave, and placed within a spit inside of a convection over. The reactor was heated at 150° C. for 2 days under tumbling conditions (about 30 rpm). The product was isolated by filtration, rinsed with deionized water, and dried.

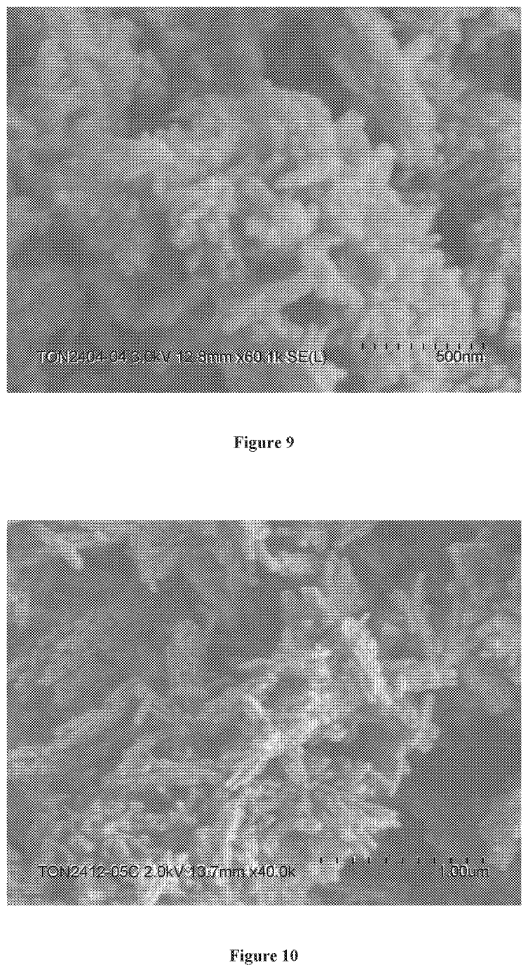

XRD analysis of the as-synthesized material showed the material to have a powder XRD pattern of ZSM-22 or TON framework type. FIG. 9 is a SEM image of the as-synthesized product and indicates that the crystals have a uniform rice morphology (L/D≤3) with a particle length of from 50 to 100 nm.

### Example 16: 1,3-dimethylimidazolium chloride

This Example was conducted in similar conditions as Example 15 except for a higher amount of water, to produce a synthesis mixture having the following composition in terms of molar ratios:
20 H₂O: 1 SiO₂: 0.0125 Al₂O₃: 0.05 QCl: 0.2 NaOH

After 2 days of heating at 160° C., a molecular sieve of TON framework type or ZSM-22 was obtained, as identified by its XRD pattern.

### Example 17: 1,3-dimethylimidazolium chloride

This Example was conducted in similar conditions as Example 15 except for a silica to alumina molar ration of 200 and a lower amount of NaOH, to produce a synthesis mixture having the following composition in terms of molar ratios:
15 H₂O: 1 SiO₂: 0.005 Al₂O₃: 0.05 QCl: 0.14 NaOH

After 2 days of heating at 150° C., a molecular sieve of TON framework type or ZSM-22 was obtained, as identified by its XRD pattern.

### Example 18: 1,3-dimethylimidazolium chloride

This Example was conducted in similar conditions as Example 10, except for a silica to alumina molar ratio of 66.7, to produce a synthesis mixture having the following composition in terms of molar ratios:
17 H₂O: 1 SiO₂: 0.015 Al₂O₃: 0.05 QCl: 0.2 KOH

After 2 days of heating at 160° C., a molecular sieve of TON framework type or ZSM-22 was obtained, as identified by its XRD pattern.

### Example 19: 1,3-dimethylimidazolium chloride—No Seeds

In a PTFE liner for a 23 mL steel Parr autoclave, the following were mixed: 0.88 g of 1,3-dimethylimidazolium chloride solution (purchased from TCI, 20 wt %), 1.39 g of NaOH solution (10 wt %), 0.24 g of sodium aluminate (NaAlO₂, 23.5 wt % Al₂O₃, 19.4 wt % Na₂O), 6.9 g of deionized water, without adding any ZSM-22 seeds, and 1.59 g of precipitated silica (Sipernat® 340 available from Evonik), to produce a synthesis mixture having the following composition in terms of molar ratios:
20 H₂O: 1 SiO₂: 0.022 Al₂O₃: 0.05 QCl: 0.2 NaOH

The liner was then capped, sealed within a 23 mL Parr autoclave, and placed within a spit inside of a convection oven. The reactor was heated at 160° C. for 2 days under tumbling conditions (about 30 rpm). The product was isolated by filtration, rinsed with deionized water, and dried.

XRD analysis of the as-synthesized material showed the material to have a powder XRD pattern of ZSM-22 or TON framework type. FIG. 10 shows a SEM image of the as-synthesized material and indicates that the crystals have a uniform rice morphology (L/D≤3) with an average particle length of about 200 nm.

### Example 20: 1,3-dimethylimidazolium chloride—No Seeds

In a PTFE liner for a 23 mL steel Parr autoclave, the following were mixed: 1.1 g of 1,3-dimethylimidazolium chloride solution (purchased from TCI, 20 wt %), 1.86 g of NaOH solution (10 wt %), 0.32 g of sodium aluminate (NaAlO₂, 23.5 wt % Al₂O₃, 19.4 wt % Na₂O), 6.2 g of deionized water, without adding any ZSM-22 seeds, and 2 g of precipitated silica (Sipernat® 340 available from Evonik), to produce a synthesis mixture having the following composition in terms of molar ratios:
15 H₂O: 1 SiO₂: 0.022 Al₂O₃: 0.05 QCl: 0.2 NaOH

The liner was then capped, sealed within a 23 mL Parr autoclave, and placed within a spit inside of a convection oven. The reactor was heated at 160° C. for 2 days under tumbling conditions (about 30 rpm). The product was isolated by filtration, rinsed with deionized water, and dried.

XRD analysis of the as-synthesized material showed the material to have a powder XRD pattern of ZSM-22 or TON framework type. FIG. 11 shows a SEM image of the as-synthesized material and indicates that the crystals have a uniform rice morphology (L/D<3) with an average particle length of about 100 nm.

### Comparative Example 21: 1,3-dimethylimidazolium hydroxide

1,3-dimethylimidazolium chloride (purchased from TCI) was ion-exchanged with ion-exchange resin Amberlite® IRN78 OH hydroxide form, with a chloride:resin:water ratio of 1:3:5, to its hydroxide form. The exchange was performed at room temperature overnight.

In a PTFE liner for a 23 mL Steel Parr autoclave, the following were mixed: 1.2 g of 1,3-dimethylimidazolium hydroxide solution (12.5 wt %), 0.24 g of NaOH solution (10 wt %), 2.6 g of deionized water, and 0.54 g of Ultrastable Y (USY) zeolite with a Si/Al₂ molar ratio of 60 (available from Zeolyst as CBV760), to produce a synthesis mixture having the following composition in terms of molar ratios:
25 H₂O: 1 SiO₂: 0.0167 Al₂O₃: 0.15 QOH: 0.07 NaOH

The liner was then capped, sealed within a 23 mL Parr autoclave and placed within a spit inside of a convection oven. The reactor was heated at 175° C. for 12 days under tumbling conditions (about 30 rpm). The product was isolated by filtration, rinsed with deionized water, and dried.

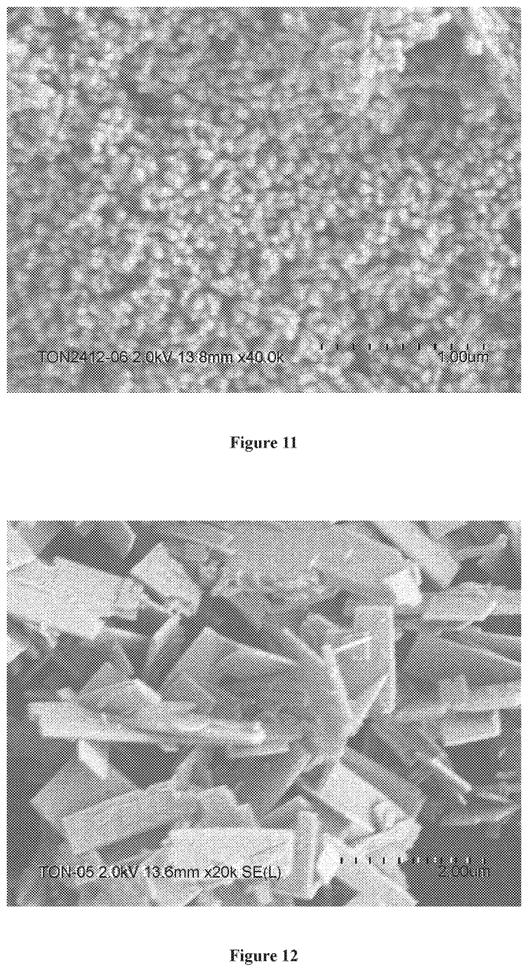

XRD analysis of the as-synthesized product showed the material to have a powder XRD pattern of ZSM-22 or TON framework type. FIG. 12 shows a SEM image of the as-synthesized product and indicates that the crystals have plate-like or rectangular-like morphology with an average particle length of about 2 microns.

### Comparative Example 22: 1,3-dimethylimidazolium hydroxide

Comparative Example 22 was conducted in similar conditions as Comparative Example 21 but reproducing the conditions and molar ratios of example “Si/Al=30, CBV 760, no seeds” of Schmidt et al., Chemistry of Materials, 27, 3756-3762 (2015).

In a PTFE liner for a 23 mL Steel Parr autoclave, the following were mixed: 4 g of 1,3-dimethylimidazolium hydroxide solution (11.3 wt %), 0.52 g of NaOH solution (10 wt %), 5.2 g of deionized water, and 1.2 g of Ultrastable Y (USY) zeolite with a Si/Al₂ molar ratio of 60 (available from Zeolyst as CBV760), to produce a synthesis mixture having the following composition in terms of molar ratios:
27.7 H₂O: 1 SiO₂: 0.0167 Al₂O₃: 0.21 QOH: 0.07 NaOH

The liner was then capped, sealed within a 23 mL Parr autoclave and placed within a spit inside of a convection oven. The reactor was heated at 175° C. for 12 days under tumbling conditions (about 30 rpm). The product was isolated by filtration, rinsed with deionized water, and dried.

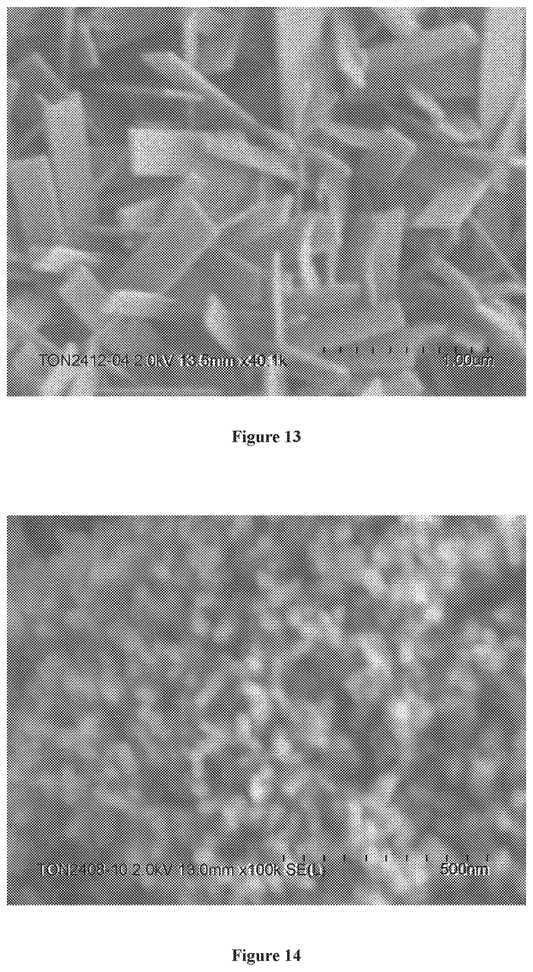

XRD analysis of the as-synthesized product showed the material to have a powder XRD pattern of ZSM-22 or TON framework type. FIG. 13 shows a SEM image of the as-synthesized product and indicates that the crystals have a plate-like or rectangular-like morphology with an average particle length of about 1 micron or longer.

### Example 23: 1-ethyl-3-methylimidazolium chloride (or 1-methyl-3-ethylimidazolium chloride)

In a PTFE liner for a 23 mL Steel Parr autoclave, the following were mixed: 1.1 g of 1-ethyl-3-methylimidazolium chloride solution (33 wt %), 1.59 g of NaOH solution (10 wt %), 0.24 g of sodium aluminate (NaAlO₂, 23.5 wt % Al₂O₃, 19.4 wt % Na₂O), 4.2 g of deionized water, 150 mg of ZSM-22 seeds, and 1.5 g of precipitated silica (Sipernat® 340 available from Evonik), to produce a synthesis mixture having the following composition in terms of molar ratios:
20 H₂O: 1 SiO₂: 0.022 Al₂O₃: 0.1 QCl: 0.22 NaOH

The liner was then capped, sealed within a 23 mL Parr autoclave and placed within a spit inside of a convection oven. The reactor was heated at 160° C. for 3 days under tumbling conditions (about 30 rpm). The product was isolated by filtration, rinsed with deionized water, and dried.

XRD analysis of the as-synthesized product showed the material to have a powder XRD pattern of ZSM-22 or TON framework type. FIG. 14 shows a SEM image of the as-synthesized product and indicates that the crystals have a uniform rice morphology (L/D<3) with an average particle length of about 100 nm.

### Example 24: 1-ethyl-3-methylimidazolium chloride (or 1-methyl-3-ethylimidazolium chloride)

In a PTFE liner for a 23 mL Steel Parr autoclave, the following were mixed: 1.1 g of 1-ethyl-3-methylimidazolium chloride solution (33 wt %), 1.11 g of KOH solution (20 wt %), 0.24 g of sodium aluminate (NaAlO₂, 23.5 wt % Al₂O₃, 19.4 wt % Na₂O), 4.75 g of deionized water, 150 mg of ZSM-22 seeds, and 1.5 g of precipitated silica (Sipernat® 340 available from Evonik), to produce a synthesis mixture having the following composition in terms of molar ratios:
20 H₂O: 1 SiO₂: 0.022 Al₂O₃: 0.1 QCl: 0.064 NaOH: 0.16 KOH

The liner was then capped, sealed within a 23 mL Parr autoclave and placed within a spit inside of a convection oven. The reactor was heated at 160° C. for 3 days under tumbling conditions (about 30 rpm). The product was isolated by filtration, rinsed with deionized water, and dried.

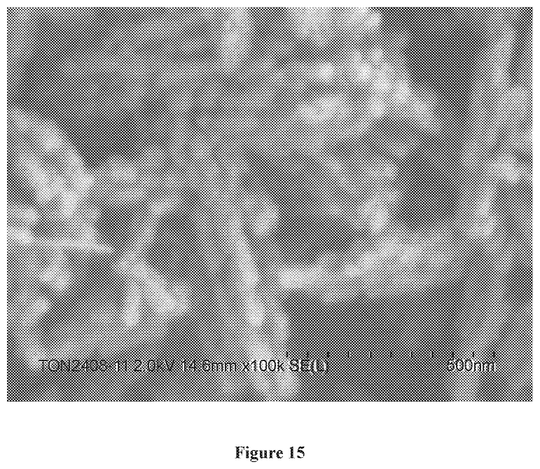

XRD analysis of the as-synthesized product showed the material to have a powder XRD pattern of ZSM-22 or TON framework type. FIG. 15 shows a SEM image of the as-synthesized product and indicates that the crystals have a uniform rice morphology (L/D≤3) with an average particle length of about 100 nm.

### Example 25: Catalyst Performance Testing in the Oligomerization of C₃-C₆ Olefins

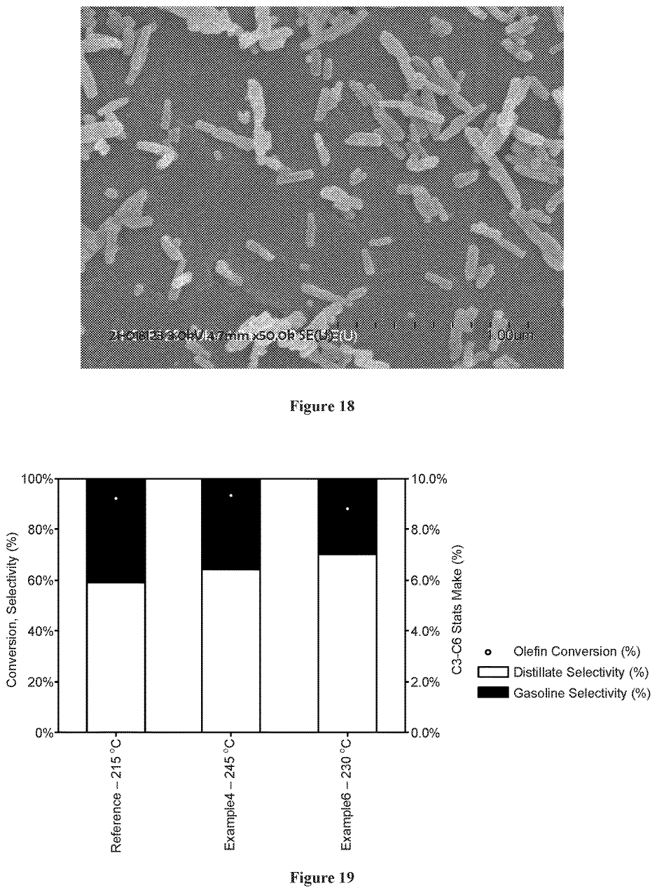

The performance of catalysts based on the ZSM-22 materials of Examples 4 and 6 and a ZSM-22 Reference material were tested in the oligomerization reaction of a C₃-C₆ olefins feed as detailed below. The Reference material was a ZSM-22 molecular sieve prepared following similar conditions as Example 1 of U.S. Pat. No. 4,902,406 but using hexamethylenediamine as the structure directing agent. FIG. 18 shows a SEM images of the Reference material and indicates that the crystals have an elongated, rod-like morphology (L/D>3) with a particle length from about 100 to 400 nm. The properties of these ZSM-22 materials are summarized in Table 1 below.

TABLE 1

| | | Particle | Aspect | |
|---|---|---|---|---|
| | SiO₂/Al₂O₃ | size | Ratio | Sₑₓₜ |
| | molar ratio | range (nm) | (L/D) | (m²/g) |
| Reference material | 74 | 100-400 | >3 | not measured |
| Example 4 | 38 | 50-100 | ≤3 | 46 |
| Example 6 | 40 | 50-100 | ≤3 | 178 |

Catalyst performance testing experiments were run using ⅓ inch (8.5 mm) internal diameter stainless steel reactor. In a typical experiment, 1.5 g of sieved solid acid catalyst (300-600 micron particle size) was loaded in a ~150 mm isothermal zone and diluted with 2-3 g SiCₓ for improved heat transfer. Indeed, olefin oligomerization is a very exothermic reaction and controlling the heat balance is important in order to measure catalyst activity accurately and to limit heavy hydrocarbon (coke) by-product formation, in order to achieve maximum catalyst life.

Typical reaction conditions were 900-1000 psig (60-70 barg), between 160° C. and 310° C., and a weight-hourly-space-velocity (WHSV) of 4 h⁻¹. Typical feed used in the experiments was 65 wt % propylene, 17 wt % 1-butene, 8 wt % 1-pentene, 3 wt % 1-hexene, 1 wt % isobutylene and ~6 wt % of C₄ saturates (isobutane and n-butane). Throughout the runs, the temperature was adjusted to achieve ~95 wt % total olefin conversion. Feed and reactor product analysis were performed by high pressure into a dedicated online gas chromatograph equipped with two low thermal mass module columns, one for light olefin/paraffin (conversion calculation) and one for heavy hydrocarbon (C₂ to C₁₆₊ carbon number distribution (CND)) analysis. To simplify CND analysis, products were fully hydrogenated online before isomer analysis using a Pt/Al₂O₃ catalyst. Distillate (i.e., jet and diesel range molecules) selectivity was defined at 50 wt % of C₉+C₁₀-C₁₆₊ selectivity. Gasoline selectivity was defined at C₆-C₈ selectivity+50 wt % of C₉ selectivity.

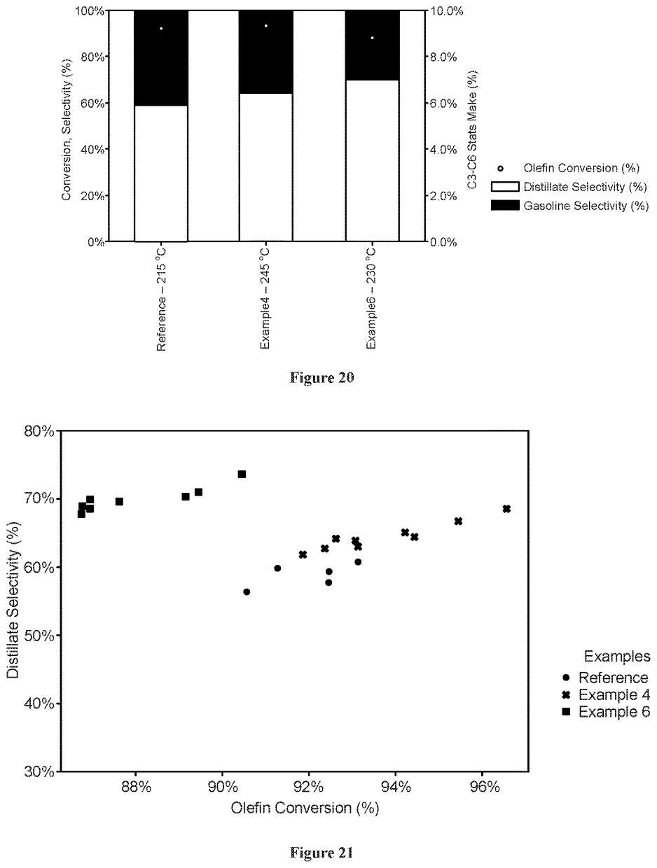

FIGS. 19 and 20 show the performance testing, in terms of conversion and selectivity, of the catalysts based on the Reference material and the materials of Examples 4 and 6. FIG. 21 shows the distillate selectivity vs. olefin conversion of the catalysts based on the Reference material and the materials of Examples 4 and 6.

Overall, the smaller, rice-shaped (L/D≤3) ZSM-22 materials of inventive Examples 4 and 6 showed an improved distillate range selectivity over the bigger, more elongated crystals of the Reference material. This effect was even more pronounced for Example 6 vs. 4, Example 6 being characterized by a higher external surface area. C₆ and C₇ selectivity (dimerization) products were also significantly suppressed over trimerization and heavier products, illustrating that the unique morphology and high external specific surface area of the innovative examples are beneficial for converting olefinic feedstock to distillate range products.

### Example 26: Catalyst Performance Testing in the Oligomerization of C₂-C₆ Olefins

The performance of catalysts based on the ZSM-22 materials of Examples 4 and 6 and the Reference material, as described under Example 25, were tested in the oligomerization reaction of a C₂-C₆ olefins feed. Equipment and conditions were similar as in Example 25, except for a feed comprising 18-20 wt % ethylene, 53-55 wt % propylene, 15-17 wt % 1-butene, 6-7 wt % 1-pentene, 2-3 wt % 1-hexene, 1-2 wt % isobutylene and −1 wt % isobutane.

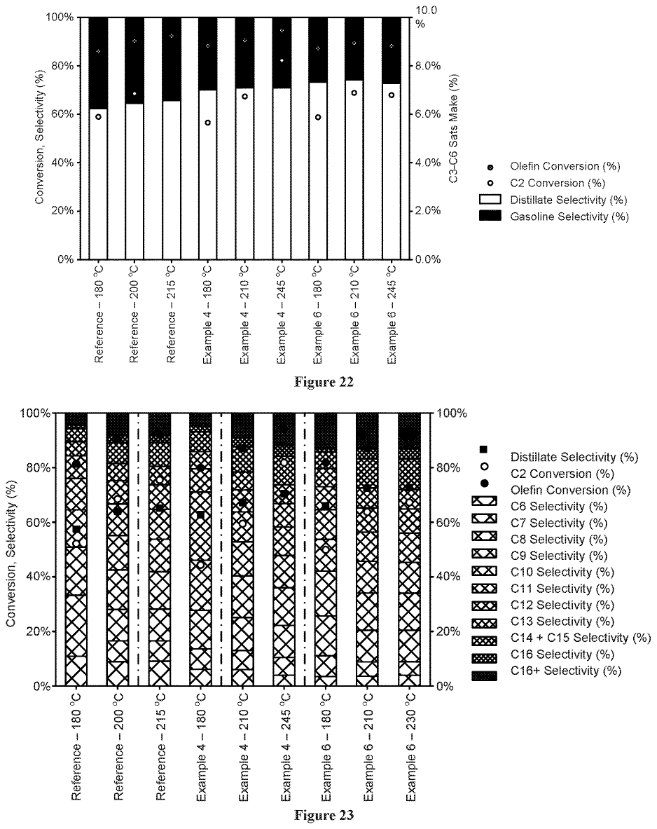

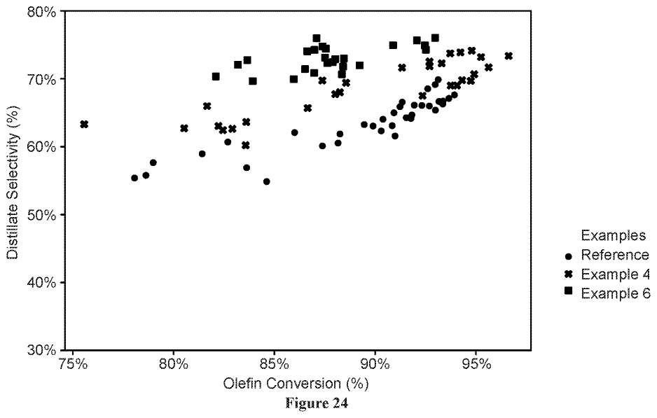

FIGS. 22 and 23 show the performance testing, in terms of conversion and selectivity at various temperatures, of the catalysts based on the Reference material and the materials of Examples 4 and 6. FIG. 24 shows the distillate selectivity vs. olefin conversion of the catalysts based on the Reference material and the materials of Examples 4 and 6.

Overall, the smaller, rice-shaped (L/D≤3) ZSM-22 materials of inventive Examples 4 and 6 showed an improved distillate range selectivity over the bigger, more elongated crystals of the Reference material. The materials of Examples 4 and 6 also showed a higher distillate selectivity over a broader olefin conversion range as compared to the Reference material, especially for Example 6 which is characterized by a higher external surface area. C₆ and C₇ selectivity (dimerization) products were also significantly suppressed over trimerization and heavier products, illustrating that the unique morphology and high external specific surface area of the innovative examples are beneficial for converting olefinic feedstock to distillate range products.

While the present invention has been described and illustrated with reference to particular embodiments, it will be appreciated by those of ordinary skill in the art that the invention lends itself to many different alterations, modifications, and variations not specifically illustrated herein. It will also be apparent to those skilled in the art that when numerical lower limits and numerical upper limits are listed herein, ranges from any lower limit to any upper limit are contemplated. Also, all numerical values within the detailed description herein are modified by “about” the indicated value, and take into account experimental error and variations that would be expected by a person having ordinary skill in the art.

Where in the foregoing description, integers or elements are mentioned which have known, obvious or foreseeable equivalents, then such equivalents are herein incorporated as if individually set forth. Reference should be made to the claims for determining the true scope of the present invention, which should be construed so as to encompass any such equivalents. It will also be appreciated by the reader that integers or features of the invention that are described as preferable, advantageous, convenient or the like are optional and do not limit the scope of the independent claims. Moreover, it is to be understood that such optional integers or features, whilst of possible benefit in some embodiments of the invention, may not be desirable, and may therefore be absent, in other embodiments.

Additionally or alternately, the invention relates to:

Embodiment 1: A method of making an aluminum-containing molecular sieve of TON framework type, comprising the steps of:
(a) preparing a synthesis mixture comprising water, a source of silica, a source of alumina, a structure directing agent (Q) comprising at least one cation selected from 1,3-dimethylimidazolium cations and 1-ethyl-3-methylimidazolium cations, a source of hydroxide ions (OH), a source of alkali and/or alkaline earth metal element (M), and a source of halide ions (W) different from fluoride ions;
(b) heating said synthesis mixture under crystallization conditions including a temperature of from 100 to 200° C. for a time sufficient to form crystals of said molecular sieve; and
(c) recovering at least a portion of the molecular sieve from step (b).

Embodiment 2: The method of Embodiment 1, further comprising treating the molecular sieve recovered in step (c) to remove at least part of the structure directing agent (Q).

Embodiment 3: The method of Embodiment 1 or 2, wherein the structure directing agent (Q) is in the form of a halide, hydroxide or nitrate, preferably wherein the structure directing agent (Q) is in halide form, in particular in its chloride or bromide form, more preferably its chloride form.

Embodiment 4: The method of any one of Embodiments 1 to 3, wherein the synthesis mixture has the following composition in terms of molar ratios:

| Molar | Typical | Preferred | More preferred |
|---|---|---|---|
| ratios | range | range | range |
| Si/Al₂ | 25-300 | >30-250 | 35-200 |
| Q/Si | 0.01-1.0 | 0.02-0.7 | 0.03-0.3 |
| OH/Si | 0.01-1.0 | 0.05-0.8 | 0.1-0.5 |
| M/Si | 0.01-1.0 | 0.05-0.8 | 0.1-0.5 |
| W/Si | 0.01-1.0 | 0.02-0.7 | 0.03-0.5 |
| H₂O/Si | 1-100 | 5-80 | 10-50 |

Embodiment 5: The method of any one of Embodiments 1 to 4, wherein the synthesis mixture has a Q/Si molar ratio of less than 0.3, preferably less than 0.2.

Embodiment 6: The method of any one of Embodiments 1 to 5, wherein the synthesis mixture has Q/M molar ratio of less than 2.0, preferably less than 1.0, more preferably less than 0.8.

Embodiment 7: The method of any one of Embodiments 1 to 6, wherein the synthesis mixture has a F/Si molar ratio of less than 0.05, in particular less than 0.01, more particularly less than 0.005, most particularly wherein the synthesis mixture is substantially free of fluoride ions.

Embodiment 8: The method of any one of Embodiments 1 to 7, wherein the crystallization conditions include heating to a temperature of from 120° C. to less than 175° C., preferably from 140° C. to 170° C.

Embodiment 9: The method of any one of Embodiments 1 to 8, wherein the crystallization conditions include heating for a time sufficient to form crystals of said molecular sieve but for less than 10 days, more particularly for less than 7 days.

Embodiment 10: The method of any one of Embodiments 1 to 9, wherein the synthesis mixture comprises seeds, in particular seeds of TON framework type.

Embodiment 11: The method of any one of Embodiments 1 to 10, wherein the molecular sieve of TON framework type is selected from the group consisting of ISI-1, KZ-2, Nu-10, Theta-1, ZSM-22 zeolites and mixtures thereof, in particular ZSM-22.

Embodiment 12: An aluminum-containing molecular sieve of TON framework type obtainable by the method of any one of the preceding Embodiments.

Embodiment 13: An aluminum-containing molecular sieve of TON framework type, wherein the molecular sieve is in the form of crystals having a maximal particle size and/or an average maximal particle size of less than 500 nm, wherein at least 50% of the molecular sieve crystals (as vol %) have an aspect ratio (L/D) of at most 3, as determined by scanning electron microscopy (SEM).

Embodiment 14: The molecular sieve of Embodiment 12 or 13, wherein the molecular sieve is in the form of crystals having a maximal particle size and/or an average maximal particle size of less than 300 nm, preferably less than 250 nm, more preferably less than 100 nm, as determined by scanning electron microscopy (SEM).

Embodiment 15: The molecular sieve of any one of Embodiments 12 to 14, wherein the molecular sieve has a silica to alumina molar ratio of from 25 to 300, preferably from 30 or from more than 30 to 250, more preferably from 35 to 200, as determined by XRF.

Embodiment 16: The molecular sieve of any one of Embodiments 12 to 15, wherein the molecular sieve is an aluminosilicate molecular sieve.

Embodiment 17: The molecular sieve of any one of Embodiments 12 to 16, wherein the molecular sieve is selected from the group consisting of IS-1, KZ-2, Nu-10, Theta-1, ZSM-22 zeolites and mixtures thereof, in particular ZSM-22.

Embodiment 18: A process of converting an organic compound to a conversion product, which comprises contacting the organic compound with the molecular sieve of any one of Embodiments 12 to 17.

Embodiment 19: The process of Embodiment 18, that is an olefin oligomerization process.

Embodiment 20: The process of Embodiment 19, wherein an olefin feed stream containing at least one C₂-C₁₂ olefin, preferably at least one C₂-C₈ olefin, more preferably at least one C₂-C₆ olefin, is contacted with the molecular sieve of any one of Embodiments 12 to 17, to produce oligomerized products, including trimer and heavier oligomer products.

1. A method of making an aluminum-containing molecular sieve of TON framework type, comprising the steps of:
(a) preparing a synthesis mixture comprising water, a source of silica, a source of alumina, a structure directing agent (Q) comprising at least one cation selected from 1,3-dimethylimidazolium cations and 1-ethyl-3-methylimidazolium cations, a source of hydroxide ions (OH), a source of alkali and/or alkaline earth metal element (M), and a source of halide ions (W) different from fluoride ions;
(b) heating said synthesis mixture under crystallization conditions including a temperature of from 100° C. to 200° C. for a time sufficient to form crystals of said molecular sieve;
(c) recovering at least a portion of the molecular sieve from step (b); and
(d) optionally treating the molecular sieve recovered in step (c) to remove at least part of the structure directing agent (Q).

2. The method of claim 1, wherein the structure directing agent (Q) is in the form of a halide, hydroxide or nitrate.

3. The method of claim 2, wherein the structure directing agent (Q) is in chloride form or bromide form.

4. The method of claim 1, wherein the synthesis mixture has the following composition in terms of molar ratios:

| Molar ratios | |
|---|---|
| Si/Al₂ | 25-300 |
| Q/Si | 0.01-1.0 |
| OH/Si | 0.01-1.0 |
| M/Si | 0.01-1.0 |
| W/Si | 0.01-1.0 |
| H₂O/Si | 1-100 |

5. The method of claim 1, wherein the synthesis mixture has the following composition in terms of molar ratios:

| Molar ratios | |
|---|---|
| Si/Al₂ | 30-250 |
| Q/Si | 0.02-0.7 |
| OH/Si | 0.05-0.8 |
| M/Si | 0.05-0.8 |
| W/Si | 0.02-0.7 |
| H₂O/Si | 5-80 |

6. The method of claim 1, wherein the synthesis mixture has the following composition in terms of molar ratios:

| Molar ratios | |
|---|---|
| Si/Al₂ | 35-200 |
| Q/Si | 0.03-0.3 |
| OH/Si | 0.1-0.5 |
| M/Si | 0.1-0.5 |
| W/Si | 0.03-0.5 |
| H₂O/Si | 10-50 |

7. The method of claim 1, wherein the synthesis mixture has a Q/Si molar ratio of less than 0.3, a Q/M molar ratio of less than 2.0, or a combination thereof.

8. The method of claim 1, wherein the synthesis mixture has a Q/Si molar ratio of less than 0.3, a Q/M molar ratio of less than 1.0, or a combination thereof.

9. The method of claim 1, wherein the crystallization conditions include heating to a temperature of from 120° C. to less than 175° C. for a time sufficient to form crystals of said molecular sieve.

10. The method of claim 1, wherein the synthesis mixture comprises seeds, the seeds optionally being of TON framework type.

11. The method of claim 1, wherein the molecular sieve of TON framework type is selected from the group consisting of IS-1, KZ-2, Nu-10, Theta-1, ZSM-22 zeolites and mixtures thereof.

12. The method of claim 1, wherein the molecular sieve of TON framework type is ZSM-22.

13. An aluminum-containing molecular sieve of TON framework type, wherein the molecular sieve is in the form of crystals having at least one of a maximal particle size of less than 500 nm and an average maximal particle size of less than 500 nm, and wherein at least 50% of the molecular sieve crystals (as vol %) have an aspect ratio (L/D) of at most 3, as determined by scanning electron microscopy (SEM).

14. The molecular sieve of claim 13, wherein the molecular sieve is in the form of crystals having a maximal particle size and/or an average maximal particle size of less than 300 nm, preferably less than 250 nm, more preferably less than 100 nm, as determined by scanning electron microscopy (SEM).

15. The molecular sieve of claim 13, wherein the molecular sieve has a silica to alumina molar ratio of from 25 to 300, as determined by XRF.

16. The molecular sieve of claim 13, wherein the molecular sieve has a silica to alumina molar ratio of from 30 to 250, as determined by XRF.

17. The molecular sieve of claim 13, wherein the molecular sieve is an aluminosilicate molecular sieve.

18. The molecular sieve of claim 13, wherein the molecular sieve is selected from the group consisting of IS-1, KZ-2, Nu-10, Theta-1, ZSM-22 zeolites and mixtures thereof.

19. A process of converting an organic compound to a conversion product, comprising:
contacting an olefin feed stream comprising at least one C₂-C₁₂ olefin with the molecular sieve of claim 13 under olefin oligomerization conditions to form a conversion product comprising oligomerized products, the oligomerized products comprising trimer and heavier oligomer products.

20. The process of claim 19, wherein the olefin feed stream comprises at least one C₂-C₈ olefin.
